(19) United States
(12) Patent Application Publication
(10) Pub. No.: US 2017/0035795 A1
(43) Pub. Date: Feb. 9, 2017

(54) METHODS AND REAGENTS FOR THE DIAGNOSIS AND TREATMENT OF ACUTE LEUKEMIA

(71) Applicants: Iannis AIFANTIS, Brooklyn, NY (US); Thomas TRIMARCHI, Brooklyn, NY (US)

(72) Inventors: Iannis AIFANTIS, Brooklyn, NY (US); Thomas TRIMARCHI, Brooklyn, NY (US)

(73) Assignee: New York University, New York, NY (US)

(21) Appl. No.: 15/225,294

(22) Filed: Aug. 1, 2016

Related U.S. Application Data
(60) Provisional application No. 62/199,080, filed on Jul. 30, 2015.

(51) Int. Cl. A61K 31/713; G01N 33/574; A61K 45/06; C12Q 1/68

(52) U.S. Cl. CPC A61K 31/713; C12Q 1/6886; G01N 33/57426; A61K 45/06; C12Q 2600/158; C12Q 2600/178

(57) ABSTRACT

The present invention is directed to methods of treating T-ALL that involves administering a therapeutic agent that inhibits a NOTCH-1 regulated non-coding RNA (lncRNA). Another embodiment of the invention relates to methods and kits for diagnosing T-ALL that involve detecting and quantifying the expression level of NOTCH1-regulated lncRNAs is a biological sample from a subject.

This application claims priority benefit of U.S. Provisional Patent Application No. 62/199,080, filed Jul. 30,2015, which is hereby incorporated by reference in its entirety.

This invention was made with government support under grant number 5R01CA194923-03 awarded by the U.S. National Institutes of Health. The government has certain rights in this invention.

### FIELD OF THE INVENTION

The present invention relates to genome-wide mapping and characterization of notch-regulated long noncoding RNAs in acute leukemia.

### BACKGROUND OF THE INVENTION

T cell acute lymphoblastic leukemia (T-ALL) is an aggressive hematological neoplasm that results from the malignant transformation of T-lymphocyte progenitors. T-ALL accounts for only 15%-20% of all acute lymphoblastic leukemia (ALL) cases and is associated with a disproportionate amount of treatment failures and increased mortality. Although the genetic events leading to transformation in T-ALL are complex, aberrant NOTCH1 signaling is a unifying feature, with activating mutations found in more than 50% of cases (Ferrando, A.A., “The Role of NOTCH1 Signaling in T-ALL,” Hematology (Am. Soc. Hematol. Educ. Program) 353-361 (2009); Weng et al., “Activating Mutations of NOTCH1 in Human T Cell Acute Lymphoblastic Leukemia,” Science 306:269-271 (2004)). Previous efforts by our group and others have aimed at dissecting additional genetic (De Keersmaecker et al., “Exome Sequencing Identifies Mutation in CNOT3 and Ribosomal Genes RPL5 and RPL10 in T-cell Acute Lymphoblastic Leukemia,” Nat. Genet. 45:186-190 (2013); Ntziachristos et al., “Genetic Inactivation of the Polycomb Repressive Complex 2 in T Cell Acute Lymphoblastic Leukemia,” Nat. Med. 18:298-301 (2012); Zhang et al., “The Genetic Basis of Early T-cell Precursor Acute Lymphoblastic Leukaemia,” Nature 481:157-163 (2012)) and molecular (Ferrando et al., “Gene Expression Signatures Define Novel Oncogenic Pathways in T cell Acute Lymphoblastic Leukemia,” Cancer Cell 1:75-87 (2002); Look, A. T., “Molecular Pathways in T-cell Acute Lympho-blastic Leukemia: Ramifications for Therapy,” Clin. Adv. Hematol. Oncol. 2:779-780 (2004)) changes that contribute to induction of T-ALL. Such efforts have yielded important prognostic tools and a more complete understanding of the molecular basis of ALL, including the identification of T-ALL oncogenes (NOTCH1, MYC) and tumor suppressors (FBXW7, CYLD, EZH2, SUZ12) and the identification and characterization of the leukemia-initiating cell (King et al., “The Ubiquitin Ligase FBXW7 Modulates Leukemia-initiating Cell Activity by Regulating MYC Stability,” Cell 153:1552-1566 (2013)). To date, most efforts toward understanding T-ALL have focused on genetic and epigenetic alterations, which ultimately impact function of protein-coding genes. Additionally, several groups have investigated the involvement of noncoding microRNAs (miRNAs) in T-ALL (Fragoso et al., “Modulating the Strength and Threshold of NOTCH Oncogenic Signals by mir-181a-1/b-1,” PLoS Genet. 8:e1002855 (2012); Yu et al., “Microarray Detection of Multiple Recurring Submicroscopic Chromosomal Aberrations in Pediatric T-cell Acute Lymphoblastic Leukemia,” Leukemia 25:1042-1046 (2011)); however, the requirement of other classes of noncoding RNA for T cell transformation and maintenance has not been investigated thus far.

Recent evidence has revealed that a large portion of the human genome is transcriptionally active despite the fact that only a small portion contains protein-coding genes (Carninci et al., FANTOM Consortium; RIKEN Genome Exploration Research Group and Genome Science Group (Genome Network Project Core Group), “The Transcriptional Landscape of the Mammalian Genome,” Science 309:1559-1563 (2005); Clark et al., “The Reality of Pervasive Transcription,” PLoS Biol. 9:e1000625, discussion e1001102 (2011); Djebali et al., “Landscape of Transcription in Human Cells,” Nature 489:101-108 (2012); Katayama et al., RIKEN Genome Exploration Research Group; Genome Science Group (Genome Network Project Core Group); FANTOM Consortium, “Antisense Transcription in the Mammalian Transcriptome,” Science 309:1564-1566 (2005)). This observation has led many to hypothesize that both human and mouse genomes contain thousands of long noncoding RNA (lncRNA) genes (Cabili et al., “Integrative Annotation of Human Large Intergenic Noncoding RNAs Reveals Global Properties and Specific Subclasses,” Genes Dev. 25:1915-1927 (2011); Guttman et al., “Chromatin Signature Reveals Over a Thousand Highly Conserved Large Non-coding RNAs in Mammals,” Nature 458:223-227 (2009)). Although some have suggested that a subset of lncRNAs are the result of divergent transcription at protein coding gene promoters (Almada et al., “Promoter Directionality is Controlled by U1 snRNP and Polyadenylation Signals,” Nature 499:360-363 (2013); Seila et al., “Divergent Transcription from Active Promoters,” Science 322:1849-1851(2008)), this does not explain the existence of many intergenic lncRNAs that do not originate from bidirectional promoters (Cabili et al., “Integrative Annotation of Human Large Intergenic Noncoding RNAs Reveals Global Properties and Specific Subclasses,” Genes Dev. 25:1915-1927 (2011); Sigova et al., “Divergent Transcription of Long Noncoding RNA/mRNA Gene Pairs in Embryonic Stem Cells,” Proc. Natl. Acad. Sci. USA 110:2876-2881(2013)). In general, lncRNAs are a heterogeneous class of transcripts without any single unifying feature except for an arbitrary minimum length of 200 nucleotides and an apparent lack of protein-coding potential (Guttman et al., “Ribosome Profiling Provides Evidence That Large Noncoding RNAs do not Encode Proteins,” Cell 154:240-251 (2013); Rinn et al., “Genome Regulation by Long Noncoding RNAs,” Annu. Rev. Biochem. 81:145-166 (2012); Ulitsky et al., “lincRNAs: Genomics, Evolution, and Mechanisms,” Cell 154:26-46 (2013)). Despite this, lncRNAs have been shown to be important in development (Boumil et al., “Forty Years of Decoding the Silence in X-chromosome Inactivation,” Hum. Mol. Genet. 10:2225-2232 (2001); Grote et al., “The Tissue-specific lncRNA Fendrr is an Essential Regulator of Heart and Body Wall Development in the Mouse,” Dev. Cell 24:206-214 (2013); Guttman et al., “lincRNAs Act in the Circuitry Controlling Pluripotency and Differentiation,” Nature 477:295-300 (2011); Klattenhoff et al., “Braveheart, a Long Noncoding RNA Required for Cardiovascular Lineage Commitment,” Cell 152:570-583 (2013); Kretz et al., “Control of Somatic Tissue Differentiation by the Long Non-coding RNA TINCR,” Nature 493:231-235 (2013); Loewer et al., “Large Intergenic Non-coding RNA-RoR Modulates Reprogramming of Human Induced Pluripotent Stem Cells,” Nat Genet. 42:1113-1117 (2010); Rinn et al., “Functional Demarcation of Active and Silent Chromatin Domains in Human HOX Loci by Noncoding RNAs,” Cell 129:1311-1323 (2007)) and disease (Gomez et al., “The NeST Long ncRNA Controls Microbial Susceptibility and Epigenetic Activation of the Interferon-g Locus,” Cell 152:743-754 (2013); Gupta et al., “Long Non-coding RNA HOTAIR Reprograms Chromatin State to Promote Cancer Metastasis,” Nature 464:1071-1076 (2010); Huarte et al., “A Large Intergenic Noncoding RNA Induced by p53 Mediates Global Gene Repression in the p53 Response,” Cell 142:409-419 (2012); Lee et al., “Transcriptome Sequencing in Sezary Syndrome Identifies Sezary Cell and Mycosis Fungoides Associated lncRNAs and Novel Transcripts,” Blood 120:3288-3297 (2012); Yildirim et al., “Xist RNA is a Potent Suppressor of Hematologic Cancer in Mice,” Cell 152:727-742 (2013)) in many different cell types, suggesting a ubiquitous role in regulation of cellular state. Although the molecular mechanisms by which lncRNAs act are poorly understood, they have been implicated as regulators of diverse cellular processes including regulation of cell cycle (Hung et al., “Extensive and Coordinated Transcription of Noncoding RNAs Within Cell-cycle Promoters,” Nat. Genet. 43:621-629 (2011)), RNA stability (Kretz et al., “Control of Somatic Tissue Differentiation by the Long Non-coding RNA TINCR,” Nature 493:231-235 (2013)), and chromatin structure (Gupta et al., “Long Non-coding RNA HOTAIR Reprograms Chromatin State to Promote Cancer Metastasis,” Nature 464:1071-1076 (2010); Lee, J. T., “Epigenetic Regulation by Long Noncoding RNAs,” Science 338:1435-1439 (2012); Tsai et al., “Long Noncoding RNA as Modular Scaffold of Histone Modification Complexes,” Science 329:689-693 (2010); Wang et al., “A Long Noncoding RNA Maintains Active Chromatin to Coordinate Homeotic Gene Expression,” Nature 472:120-124 (2011b); Yang et al., “ncRNA- and Pc2 Methylation-dependent Gene Relocation Between Nuclear Structures Mediates Gene Activation Programs,” Cell 147:773-788 (2011)). Thus, further efforts toward accurate annotation and functional significance of lncRNAs will be critical for our understanding of these processes.

The present invention is directed to overcoming these and other deficiencies in the art.

### SUMMARY OF THE INVENTION

A first aspect of the present invention relates to a method of treating T cell acute lymphoblastic leukemia in a subject. This method involves selecting a subject having T cell acute lymphoblastic leukemia and administering, to the subject, a therapeutic agent that inhibits a NOTCH-1 regulated long non-coding RNA (lncRNA) at a dosage effective to treat the T cell acute lymphoblastic leukemia in the subject.

Another aspect of the present invention relates to a method of diagnosing T cell acute lymphoblastic leukemia in a subject. This method involves measuring, in a biological sample obtained from a subject, expression levels of one or more NOTCH-1 regulated lncRNA transcripts and comparing the measured expression levels to control expression levels of the one or more NOTCH-1 regulated lncRNA transcripts. The subject is then diagnosed as having T cell acute lymphoblastic leukemia based on the comparing.

Another aspect of the present invention is directed to a kit suitable for diagnosing T-ALL. This kit includes one or more reagents suitable for detecting the expression levels of one or more NOTCH-1 regulated lncRNAs.

Although several groups have investigated possible roles for lncRNAs as players in the TP53 tumor-suppressor transcriptional program (Huarte et al., “A Large Intergenic Noncoding RNA Induced by p53 Mediates Global Gene Repression in the p53 Response,” Cell 142:409-419 (2012); Hung et al., “Extensive and Coordinated Transcription of Noncoding RNAs Within Cell-cycle Promoters,” Nat. Genet. 43:621-629 (2011)) and solid tumors (Du et al., “Integrative Genomic Analyses Reveal Clinically Relevant Long Noncoding RNAs in Human Cancer,” Nat. Struct Mol. Biol. 20:908-913 (2013); Prensner et al., “Transcriptome Sequencing Across a Prostate Cancer Cohort Identifies PCAT-1, an Unannotated lincRNA Implicated in Disease Progression,” Nat. Biotechnol. 29:742-749 (2011); Prensner et al., “The Long Noncoding RNA SChLAP1 Promotes Aggressive Prostate Cancer and Antagonizes the SWI/SNF Complex,” Nat. Genet. 45:1392-1398 (2013); Yang et al., “lncRNA-dependent Mechanisms of Androgen-receptor-regulated Gene Activation Programs,” Nature 500:598-602 (2013)), the overall knowledge of lncRNAs in cancer, including leukemia, remains extremely limited (Garding et al., “Epigenetic Upregulation of lncRNAs at 13q14.3 in Leukemia is Linked to the In Cis Downregulation of a Gene Cluster That Targets NF-kB. PLoS Genet,” 9:e1003373 (2013); Lee, J. T., “Epigenetic Regulation by Long Noncoding RNAs,” Science 338:1435-1439 (2012)). Here, multiple genome-wide data sets were used in order to create the first all-inclusive lncRNA annotation and mapping in human T-ALL. This annotation was used to examine lncRNA expression profiles in the context of oncogenic NOTCH1 signaling and identify Notch-dependent lncRNA expression programs that are deregulated in T-ALL. Finally, functional studies were used to identify T-ALL-specific activator lncRNAs that have a key role in promoting tumor maintenance. Altogether these studies demonstrate lncRNAs are key regulators of the pathogenic state in acute leukemia and carry important clinical relevance as biomarkers or therapeutic targets.

### BRIEF DESCRIPTION OF DRAWINGS

FIGS. 1A-1E show the long noncoding transcriptome in T-ALL. FIG. 1A shows a schematic illustration of the procedure used to discover and define lncRNAs in T-ALL. FIG. 1B shows a venn diagram depicting the overlap between our catalog of T-ALL-associated lncRNAs and those in the Gencode v18 lncRNA collection. FIG. 1C shows a pie chart representation showing the proportion of T-ALL-associated lncRNAs that are transcribed in a divergent orientation (red) or intergenic (blue) with respect to protein-coding genes (green, diagram left). FIG. 1D shows a violin plot of log 2 maximum expression values (FPKM) for protein-coding (red) and T-ALL lncRNA (blue) genes. Boxes represent first and third quartiles. Whiskers are 1.5 times the interquartile range (IQR). FIG. 1E shows a heatmap representation of the 250 lncRNAs with the most variable (IQR/median) expression across T-ALL, normal T cells, and various other somatic tissues.

FIGS. 2A-2D show T-ALL-associated lncRNAs have low coding potential and are enriched in the nucleus. FIG. 2A shows a cumulative frequency plot of PhyloCSF scores for lncRNAs (blue) and RefSeqNM entries (red). FIG. 2B shows a heatmap representation of lncRNA expression in whole cell RNA extracts compared to nuclear RNA extract. FIG. 2C shows bar plots showing the proportion of T-ALL lncRNAs that are included in GENCODE. FIG. 2D shows bar plots showing the proportion of divergent (yellow) versus intergenic (blue) lncRNAs that are active (FPKM>1) in primary T-ALL (left) compared to thymus (right).

FIGS. 3A-3H show lncRNAs are a component of the Notch1 transcriptional network. FIG. 2A shows a heatmap representation of ChIP-seq signal density for H3K27ac, H3K4me3, RNAP2, NOTCH1, RBPJk, and ZNF143 centered on lncRNA TSSs±5 kb. All ChIP experiments are from CUTLL1 cells. FIG. 3B shows a histogram depicting ChIP-seq signal density on all T-ALL lncRNA promoters for H3K4me3, RNAP2, and NOTCH1. FIG. 3C shows scatterplots showing both lncRNA (green) and protein-coding (orange) genes whose expression is significantly altered following addition of g-SI in CUTLL1 (left) and HPBALL (right) cells. FIG. 3D shows a venn diagram showing the proportion of Notch-occupied lncRNAs whose expression is significantly downregulated in response to g-SI treatment. FIG. 3E shows GSEA enrichment plot showing significant downregulation of lncRNAs associated with the top 1,000 NOTCH1 peaks upon g-SI treatment. FIG. 3F shows heatmaps showing top differentially expressed protein-coding (left) and lncRNA (right) genes in primary T-ALL compared to thymic progenitors. FIG. 3G shows expression profiles of lncRNAs that are differentially expressed in response to g-SI treatment in T-ALL are shown in heatmap either in T-ALL (left) or CLL (right). Gray indicates no detectable expression. FIG. 3H shows expression fold-change (DMSO/g-SI) in T-ALL (CUTLL1 and HPBALL) and CLL of all lncRNAs consistently regulated upon Notch inhibition in T-ALL.

FIGS. 4A-4D show lncRNAs in the T-ALL transcriptional circuitry. FIG. 4A shows an immunoblot for NOTCH1 and qPCR for HES1, TUG1, and a panel of T-ALL-associated lncRNAs following g-SI treatment. FIG. 4B shows gene track of three Notch-dependent lncRNA loci, which show occupancy by both NOTCH1 and RBPJk at their regulatory elements (highlighted yellow). FIG. 4C shows a Venn diagram depicting lncRNAs that showed occupancy by NOTCH1 (red), ZNF143 (blue) and were downregulated upon Notch inhibition (yellow). FIG. 4D shows a heatmap representation of T-ALL-associated lncRNAs that are differentially expressed between T-ALL subtypes. Error bars represent SEM of three experiments.

FIGS. 5A-5E show LUNAR1 is a lncRNA gene controlled by NOTCH1 in T-ALL. FIG. 5A shows a correlation density plot showing expression correlation of protein-coding (red) or lncRNA (blue) genes with the nearest coding neighbor. FIG. 5B shows RNA-seq expression values for LUNAR1 in primary T-ALL and thymic progenitors (left). Overexpression was validated by qPCR (right, top), and overactivation of Notch signaling was verified by measuring HES1 expression (bottom, right). FIG. 5C shows qPCR for LUNAR1 following treatment with vehicle (blue) or g-SI (red) in CUTLL1 (left) and HPBALL (right) cells. FIG. 5D shows qPCR for LUNAR1 in Notch wild-type (WT) versus Notch mutant tumors. FIG. 5E shows immunoblot (left) for lamin B and tubulin on cytoplasmic and nuclear fractions from CUTLL1 cells. qPCR (right) for LUNAR1, U1, and GAPDH from RNA extracted from cytoplasmic and nuclear fractions. * indicates p value<0.05. Error bars represent SEM of three experiments.

FIGS. 6A-6B shows LUNAR1 chromatin state across diverse tissues. FIG. 6A shows gene track of LUNAR1 locus depicting H3K36me3 (green), H3K79me2 (blue), H3K4me1 (gray), and H3K4me3 (black) in DND41 cells. FIG. 6B shows gene track image of ChromHMM states at the LUNAR1 locus in all tissues available from the Roadmap Epigenomics project (http://www.roadmapepigenomics.org/).

FIGS. 7A-7C shows LUNAR1 is physically associated with a nearby notch-occupied enhancer. FIG. 7A shows gene track of a 2 Mb region surrounding the LUNAR1 locus including Hi-C interaction density heatmap (red upper panel), ChIP-seq tracks for H3K27ac, H3K4me1, H3K4me3, MED1, BRD4, P300, NOTCH1, RBPJk, and RNAP2, and RNA-seq track.

FIG. 7B shows gene track view of an approximately 150 kb region that contains LUNAR1 (highlighted yellow, right) and a Notch-occupied enhancer in the last intron of IGF (highlighted yellow, left). FIG. 7C shows the relative crosslinking frequency as measured by 3C-qPCR using a constant primer in a HindIII fragment at the LUNAR1 TSS (top) or at the Notch-occupied enhancer (bottom). Crosslinking frequency is relative to a negative region (green). Error bars indicate the ±SEM of three experiments.

FIGS. 8A-8C show Notch1 occupies an enhancer in the IGF1R gene. FIG. 8A shows a ChIP assay for NOTCH1, MED1, BRD4, and H3K27ac followed by qPCR for a region in the last intron of IGF1R. FIG. 8B shows a schematic of reporter constructs generated. FIG. 8C shows relative reporter activity of constructs containing the IGF1R enhancer (blue) or promoter only (yellow) upon cotransfection of ICN1. Error bars represent SEM of at least three experiments. * indicates p value<0.05.

FIGS. 9A-9F show LUNAR1 regulates T-ALL proliferation by enhancing IGF1R expression. FIG. 9A shows qPCR showing expression of LUNAR1 (blue) and IGF1R (yellow) in the presence of shRNAs targeting Renilla or LUNAR1. FIG. 9B shows line graphs showing relative contribution from T-ALL cells expressing shRNAs against LUNAR1 grown in competition with cells expressing a non-targeting shRNA. FIG. 9C is an illustration describing in vivo xenograft competition assay. FIG. 9D shows the ratio of mean fluorescence intensity (MFI) on day 28:day 0. FIG. 9E shows line graphs showing relative cell number (targeting/Scr) for cells expressing exogenous IGF1R (blue) or empty vector (red) treated with ASO. FIG. 9F shows genome-wide measurement of differentially expressed genes following pharmacological inhibition of IGF1R or LUNAR1 knockdown. Error bars represent ±SEM of at least three experiments. * indicates p value<0.05. ** indicates p value<0.01.

FIGS. 10A-10F show LUNAR1 is required for efficient T-ALL growth. FIG. 10A shows qPCR for LUNAR1 (blue) and IGF1R (yellow) in CUTLL1 cells transduced with empty vector or LUNAR1. FIG. 10B shows a line graph showing a similar competition assay as in FIG. 9B but with AML cells. FIG. 10C shows qPCR for LUNAR1 (blue) and IGF1R (yellow) in CUTLL1 cells treated with non-targeting (Scr) or LUNAR1 ASOs. FIG. 10D shows a growth curve for CUTLL1 cells treated with non-targeting (red) or LUNAR1 (blue) ASOs. FIG. 10E shows a representative FACS histogram of DNA content stained with 7AAD. Quantification percentage of CUTLL1 cells in S/G2/M phase following depletion of LUNAR1 are shown in the bar graph. FIG. 10F shows GSEA analysis with genes downregulated upon pharmacological inhibition of IGF1R (left) or targets of IGF1/2 from mSig database (right). Expression data sets used are from LUNAR1 knockdown experiments with two different shRNAs (top and bottom, respectively). Error bars represent SEM of three experiments. * indicates p value<0.05. ** indicates p value<0.01.

FIGS. 11A-11E show LUNAR1 is able to stimulate transcription of a reporter gene. FIG. 11A is an illustration describing the BoxB Gal4-1N RNA tethering system used. FIG. 11B shows a ChIP assay for Gal4-DBD at the reporter gene promoter. FIG. 11C shows luciferase reporter activity in experiments where BoxB-tagged LUNAR1 (blue) or HOTTIP (yellow) were cotransfected with Gal4-1N. FIG. 11D shows qPCR following ASO knockdown of LUNAR1 in luciferase assay. FIG. 11E shows a reporter assay showing relative reporter gene activity when BoxB-LUNAR1 was cotransfected with either non-targeting (blue) or LUNAR1-specific (yellow) ASOs. Error bars represent SEM of at least three experiments. * indicates p value<0.05. ** indicates p value<0.01.

FIGS. 12A-12F show LUNAR1 modulates mediator and RNA PolII binding at the IGF1R enhancer. FIGS. 12A-12C show ChIP assays for MED1, MED12, RNAP2, NOTCH1, H3K27ac, H3K4me1, and H3K4me3 in T-ALL cells harboring non-targeting (blue) or LUNAR1-specific shRNAs followed by locus-specific qPCR at (A) IGF1R enhancer, (B) LUNAR1 promoter, or (C) ACTB promoter. FIG. 12D shows percent recovery of LUNAR1 following ChIRP. FIG. 12E shows ChIRP assay using probes targeting LUNAR1 (blue) or LacZ (yellow) followed by qPCR at the IGF1R enhancer, LUNAR1 promoter, and ACTB promoter. FIG. 12F shows a model for cis-regulation of gene expression by LUNAR1. Error bars represent SEM of at least three experiments. * indicates p value<0.05. ** indicates p value<0.01.

### DETAILED DESCRIPTION OF INVENTION

A first aspect of the present invention relates to a method of treating T cell acute lymphoblastic leukemia in a subject. This method involves selecting a subject having T cell acute lymphoblastic leukemia and administering, to the subject, a therapeutic agent that inhibits a NOTCH-1 regulated long non-coding RNA (lncRNA) at a dosage effective to treat the T cell acute lymphoblastic leukemia in the subject.

As used herein, “subject” refers to any animal having T-cell acute lymphoblastic leukemia, which is amenable to treatment in accordance with the methods of the present invention. Preferably, the subject is a mammal. Exemplary mammalian subjects include, without limitation, humans, non-human primates, dogs, cats, rodents (e.g., mouse, rat, guinea pig), horses, cattle and cows, sheep, and pigs.

As used herein, “T-cell acute lymphoblastic leukemia” (T-ALL) refers to all subtypes and stages of the disease including, but not limited to, adult T-cell acute lymphoblastic leukemia, pediatric T-cell acute lymphoblastic leukemia, early T-cell precursor acute lymphoblastic leukemia, relapsed T-cell acute lymphoblastic leukemia, and refractory T-cell acute lymphoblastic leukemia. In one embodiment, the T-ALL is characterized by an activating mutation in NOTCH-1.

As will be appreciated, inhibitors described herein may be based on the nucleotide sequence of the target or target gene, which will be readily identifiable. Such sequences may be of mammalian origin (e.g., human or murine). In one embodiment, the therapeutic agent of the present invention inhibits a NOTCH-1 regulated lncRNA selected from the group of 130 lncRNAs listed in Table 1 below.

TABLE 1
Notch-1 Regulated lncRNAs involved in T-ALL

| | Putative |
|---|---|
| | Transcriptional |
| lncRNA | Start Site |
| XLOC_tallLncRNA_000018 | chr1: 840197 |
| XLOC_tallLncRNA_000049 | chr1: 1182710 |
| XLOC_tallLncRNA_006445 | chr1: 142618824 |
| XLOC_tallLncRNA_010256 | chr1: 205410363 |
| XLOC_tallLncRNA_012730 | chr1: 3539434 |
| XLOC_tallLncRNA_013444 | chr1: 20509388 |
| XLOC_tallLncRNA_013627 | chr1: 25370063 |
| XLOC_tallLncRNA_019127 | chr1: 146541630 |
| XLOC_tallLncRNA_022519 | chr1: 205250659 |
| XLOC_tallLncRNA_023828 | chr1: 229211784 |
| XLOC_tallLncRNA_025396 | chr10: 3985172 |
| XLOC_tallLncRNA_027390 | chr10: 39087960 |
| XLOC_tallLncRNA_032793 | chr10: 4021464 |
| XLOC_tallLncRNA_033116 | chr10: 9031433 |
| XLOC_tallLncRNA_034138 | chr10: 33410087 |
| XLOC_tallLncRNA_037253 | chr10: 106110877 |
| XLOC_tallLncRNA_042741 | chr11: 67452899 |
| XLOC_tallLncRNA_047409 | chr11: 21661275 |
| XLOC_tallLncRNA_048108 | chr11: 35157688 |
| XLOC_tallLncRNA_049325 | chr11: 64251387 |
| XLOC_tallLncRNA_049577 | chr11: 67701000 |
| XLOC_tallLncRNA_053128 | chr12: 4014236 |
| XLOC_tallLncRNA_058755 | chr12: 92860349 |
| XLOC_tallLncRNA_060249 | chr12: 122115118 |
| XLOC_tallLncRNA_060657 | chr12: 131649555 |
| XLOC_tallLncRNA_061982 | chr12: 24743616 |
| XLOC_tallLncRNA_061987 | chr12: 24757779 |
| XLOC_tallLncRNA_064256 | chr12: 68243536 |
| XLOC_tallLncRNA_067578 | chr12: 127215246 |
| XLOC_tallLncRNA_067777 | chr12: 131951366 |
| XLOC_tallLncRNA_067831 | chr12: 133001102 |
| XLOC_tallLncRNA_068464 | chr13: 22614945 |
| XLOC_tallLncRNA_068941 | chr13: 30221770 |
| XLOC_tallLncRNA_068965 | chr13: 30509893 |
| XLOC_tallLncRNA_072596 | chr13: 84714736 |
| XLOC_tallLncRNA_074562 | chr13: 28419112 |
| XLOC_tallLncRNA_079137 | chr14: 22434762 |
| XLOC_tallLncRNA_082813 | chr14: 75761106 |
| XLOC_tallLncRNA_083485 | chr14: 88490893 |
| XLOC_tallLncRNA_084878 | chr14: 22166803 |
| XLOC_tallLncRNA_089711 | chr14: 99602290 |
| XLOC_tallLncRNA_090013 | chr14: 105541719 |
| XLOC_tallLncRNA_090729 | chr15: 24736568 |
| XLOC_tallLncRNA_093454 | chr15: 69972076 |
| XLOC_tallLncRNA_094452 | chr15: 89920928 |
| XLOC_tallLncRNA_099707 | chr16: 432240 |
| XLOC_tallLncRNA_099743 | chr16: 870523 |
| XLOC_tallLncRNA_101116 | chr16: 27279525 |
| XLOC_tallLncRNA_104175 | chr16: 1025760 |
| XLOC_tallLncRNA_104176 | chr16: 1044508 |
| XLOC_tallLncRNA_112372 | chr17: 75530528 |
| XLOC_tallLncRNA_112484 | chr17: 77776952 |
| XLOC_tallLncRNA_116562 | chr17: 77896471 |
| XLOC_tallLncRNA_120552 | chr18: 70535842 |
| XLOC_tallLncRNA_121538 | chr18: 19925107 |
| XLOC_tallLncRNA_124354 | chr18: 71382799 |
| XLOC_tallLncRNA_128204 | chr19: 52097765 |
| XLOC_tallLncRNA_132633 | chr19: 57047549 |
| XLOC_tallLncRNA_133602 | chr2: 12246671 |
| XLOC_tallLncRNA_134930 | chr2: 37826907 |
| XLOC_tallLncRNA_137686 | chr2: 85649760 |
| XLOC_tallLncRNA_139520 | chr2: 122659925 |
| XLOC_tallLncRNA_140256 | chr2: 132160473 |
| XLOC_tallLncRNA_141868 | chr2: 168149509 |
| XLOC_tallLncRNA_145933 | chr2: 237683017 |
| XLOC_tallLncRNA_146619 | chr2: 8530982 |
| XLOC_tallLncRNA_146648 | chr2: 8819860 |
| XLOC_tallLncRNA_146696 | chr2: 10179076 |
| XLOC_tallLncRNA_148704 | chr2: 43197905 |
| XLOC_tallLncRNA_148727 | chr2: 43254911 |
| XLOC_tallLncRNA_151501 | chr2: 91798016 |
| XLOC_tallLncRNA_160611 | chr20: 23800483 |
| XLOC_tallLncRNA_165817 | chr21: 10014209 |
| XLOC_tallLncRNA_167830 | chr21: 47155945 |
| XLOC_tallLncRNA_168265 | chr21: 16132921 |
| XLOC_tallLncRNA_169757 | chr21: 40377663 |
| XLOC_tallLncRNA_170152 | chr21: 47001962 |
| XLOC_tallLncRNA_170428 | chr22: 18685938 |
| XLOC_tallLncRNA_170604 | chr22: 22509136 |
| XLOC_tallLncRNA_171537 | chr22: 39463242 |
| XLOC_tallLncRNA_171820 | chr22: 45048159 |
| XLOC_tallLncRNA_172200 | chr22: 16147702 |
| XLOC_tallLncRNA_172559 | chr22: 23127997 |
| XLOC_tallLncRNA_172865 | chr22: 29832998 |
| XLOC_tallLncRNA_173572 | chr22: 42760539 |
| XLOC_tallLncRNA_183900 | chr3: 161760622 |
| XLOC_tallLncRNA_191961 | chr3: 113928568 |
| XLOC_tallLncRNA_194593 | chr3: 156799455 |
| XLOC_tallLncRNA_197371 | chr4: 9495636 |
| XLOC_tallLncRNA_199312 | chr4: 38511396 |
| XLOC_tallLncRNA_202969 | chr4: 111286097 |
| XLOC_tallLncRNA_207447 | chr4: 180980095 |
| XLOC_tallLncRNA_208098 | chr4: 1575800 |
| XLOC_tallLncRNA_211319 | chr4: 65779998 |
| XLOC_tallLncRNA_213082 | chr4: 101732069 |
| XLOC_tallLncRNA_223056 | chr5: 85961922 |
| XLOC_tallLncRNA_229802 | chr5: 180018529 |
| XLOC_tallLncRNA_230589 | chr5: 15192475 |
| XLOC_tallLncRNA_238633 | chr5: 165550382 |
| XLOC_tallLncRNA_239144 | chr5: 177227984 |
| XLOC_tallLncRNA_241441 | chr6: 27661813 |
| XLOC_tallLncRNA_245747 | chr6: 105901525 |
| XLOC_tallLncRNA_247884 | chr6: 137710718 |
| XLOC_tallLncRNA_247936 | chr6: 138119181 |
| XLOC_tallLncRNA_249852 | chr6: 5795247 |
| XLOC_tallLncRNA_251637 | chr6: 31679752 |
| XLOC_tallLncRNA_252403 | chr6: 44007467 |
| XLOC_tallLncRNA_253356 | chr6: 64089020 |
| XLOC_tallLncRNA_258731 | chr6: 156572934 |
| XLOC_tallLncRNA_260764 | chr7: 144451 |
| XLOC_tallLncRNA_263519 | chr7: 50251381 |
| XLOC_tallLncRNA_266847 | chr7: 114761850 |
| XLOC_tallLncRNA_269465 | chr7: 2544558 |
| XLOC_tallLncRNA_270190 | chr7: 17189740 |
| XLOC_tallLncRNA_271222 | chr7: 38340356 |
| XLOC_tallLncRNA_271259 | chr7: 39547545 |
| XLOC_tallLncRNA_272353 | chr7: 64348921 |
| XLOC_tallLncRNA_273978 | chr7: 96592453 |
| XLOC_tallLncRNA_274179 | chr7: 100434934 |
| XLOC_tallLncRNA_278598 | chr8: 23082733 |
| XLOC_tallLncRNA_281319 | chr8: 72753812 |
| XLOC_tallLncRNA_285127 | chr8: 142384345 |
| XLOC_tallLncRNA_290267 | chr8: 104501986 |
| XLOC_tallLncRNA_294836 | chr9: 34665131 |
| XLOC_tallLncRNA_299333 | chr9: 131998411 |
| XLOC_tallLncRNA_299338 | chr9: 132096161 |
| XLOC_tallLncRNA_299900 | chr9: 2535654 |
| XLOC_tallLncRNA_313201 | chrX: 9376102 |
| XLOC_tallLncRNA_314225 | chrX: 42743369 |
| XLOC_tallLncRNA_320361 | chrY: 23411255 |

As will be understood, inhibitors of variants and isoforms of the above-noted exemplary sequences are also encompassed. In one embodiment, such variants and isoforms include nucleotide sequences that have at least 80%, at least 85%, at least 90%, at least 95%, at least 97%, or at least 99% sequence identity to a sequence identified in Table 1.

In one embodiment, the therapeutic agent inhibits the NOTCH-1 regulated lncRNA known as LUNAR1. LUNAR1, also referred to as “Leukemia-induced Noncoding Activator RNA” is a 491-nucleotide transcript, containing four exons and a poly(A) tail. LUNAR1 is located on chromosome 15 from position 99,557,680 to position 99,585143. The nucleotide sequence of LUNAR 1 cDNA is provided below as SEQ ID NO: 1. Suitable inhibitory agents for LUNAR1 are disclosed herein.

| | |
|---|---|
| aggaatggga gagggaggag gacgccaggg gcacagggac agcctccggg gcggggcgcg | 60 |
| gcgccagcct agcccgagga tggaggctga ggccgcctgt tgagtcacag tttccctgtg | 120 |
| atgctcattt taaccagatg gcacttgctt agcagtttgg aggagaagac ctgttcccct | 180 |
| gcagccttcc atgattagac ctcaactcat gagaactctt gccttgctca caagtgggag | 240 |
| taaatgctca tagctctaga aatctcccag gaaccagaca tcacggaaac tagaaaggct | 300 |
| aagggagtcc aatcttcctc taaattggcc aacagtagag tggtggaaag agaacaagct | 360 |
| gcaaaccaga cagacccaag cttgaatctc acctctgcca agctaccaga gctcaacaaa | 420 |
| ctacagacca cgggccaaat ctgacccacc gctgcttttg taaataaaac tttattggaa | 480 |
| aaaaaaaaaa a | 491 |

In another embodiment, the therapeutic agent inhibits the NOTCH-1 regulated lncRNA Lin_CXCR4, also known as LUNAR2. The genomic sequence for Lin_CXCR4 is provided below as SEQ ID NO: 2. Suitable inhibitory agents for Lin_CXCR4 are disclosed herein.

| | |
|---|---|
| aggcctttcc tgcaacactg agctgtttct ttccttttct tttttaacca tgcaacaaaa | 60 |
| cctttattag cattttgaac aggttcagct attactgaaa cttgtaattt ctaaacttaa | 120 |
| gttggggcaa atggctatac ggcagagtaa tgccatcact gggcactgcg aatgcaagac | 180 |
| tggagaatta acagccaccc ctcaggtgca ggaccaggtg cagggttgac tctttctgga | 240 |
| tgttgtagtc agaaagagtg cggccatctt ccagctgctt gcctgcaaag atgagcctct | 300 |
| gctggtcggg gctgggggtg ggggggtgcc ttctttatcc tggatcttgg ccttcacatt | 360 |
| ttccatggtg tcactgggct ccacttccag ggtgatggtc ttgccagtca gggtcttcac | 420 |
| gaagatctgc ataccacctc tcagacacag gaccaggtgc agggtctact ctttctggat | 480 |
| gttatagtca gaatgagtgc agtcatcttc cacctgcttg actgcaaaga tgagcctctg | 540 |
| ctggtccggg gtaatgcctt ccttatcctg gatcttggcc ttcacatttt cgatggtgtc | 600 |
| cctgggctcc acttcaaggg caatggtctt gctggtaagg gtcttcacga agatctgcat | 660 |
| tttgacctgt tagcggatat gacgaggctc cgaaacacca gtcatgtcca gccacaggga | 720 |
| caccaccaca tactcaccca acaaagccag tcatccctac cactgagcta tttctatgcg | 780 |
| agttcttccc ttggccctta agctgggata aatccctgtc ttcatgcaaa gttagagaca | 840 |
| tgattagata caagatctac aatatttgtg gataaaaacc aaacagttcc ttaagaaaac | 900 |
| tacaactatt ttttttggct gacaccagag tgaaatttcc cccatttatc ccccatcagc | 960 |
| ctttggtagg agcacaaaag ctacgtggca gggcacattc cagcaccatg cccatgacac | 1020 |
| caactctcgt tcattcattc cttgacgtat ttacattcaa actccgtcct cgtttgctgc | 1080 |
| tgtgctgctg gttctggctc ca | 1102 |

In another embodiment, the therapeutic agent inhibits the NOTCH-1 regulated lncRNA linc94. Linc94 is located on chromosome 18 from positions 12739485 to 12749421. The genomic sequence for linc94 is provided below as SEQ ID NO: 3. Suitable inhibitory agents for Linc94 are disclosed herein.

| | |
|---|---|
| gcgaccttga agcggcatcc gaggagatgt ggccacgggg caggcgaccg acaccagcga | 60 |
| gtccagaggg ccagcgtgtg caccactgtg tgtctccaga gacttcagga agcagccacc | 120 |
| acgcccgagg aatgcaggaa gatggacaca cggctgggga agtacaatga aaggccaagt | 180 |
| aggcagcctg ttctcctcag atcagtcccc cacgaacact cattcccgag gactcatcca | 240 |
| atactaataa gagaatgctc ttgtttttga agaattttct gaaagccatc ctgacaaatt | 300 |
| aagtagagta tgctgaagat agtcagactt tgtttttaag aattgaatat tctggaagag | 360 |
| gctcttcagt ccaatcttta gttctctcca cagagcaaac gaagtgaagt gctgaaggcc | 420 |
| tggagcccga gctgttccca cacggactcc aggacagtta aggcagggtt gccttaacta | 480 |
| aatctctgac aactgtttct tctgtctttc tcctaaaaat ggaatgcagg ccatctattc | 540 |
| tagggaaata aaggattcta gttatgtgaa tcca | 574 |

In accordance with this aspect of the present invention an inhibitor of the NOTCH-1 regulated lncRNA is administered therapeutically to a subject having T cell acute lymphoblastic leukemia. Suitable lncRNA inhibitors can act at the DNA level or at the RNA (i.e., gene product) level. As LUNAR1, for example, is a non-coding gene, there is no protein product for this gene. If inhibition is to be achieved at the DNA level, this may be done using gene therapy to knock-out or disrupt the target gene. As used herein, a “knock-out” can be a gene knockdown or the gene can be knocked out by a mutation such as, a point mutation, an insertion, a deletion, a frameshift, or a missense mutation by techniques known in the art, including, but not limited to, retroviral gene transfer.

In one embodiment, the therapeutic agent for repressing the expression of one or more NOTCH-1 regulated lncRNAs, such as LUNAR1, Linc94, or Lin_CXCR4, or any of the lncRNAs listed in Table 1 above, is a zinc finger nuclease. Zinc-finger nucleases (ZFNs) are artificial restriction enzymes generated by fusing a zinc finger DNA-binding domain to a DNA-cleavage domain. Zinc finger domains can be engineered to target desired DNA sequences, which enable zinc-finger nucleases to target unique sequence within a complex genome (Urnov et al., “Genome Editing with Engineered Zinc Finger Nucleases,” Nat. Rev. Genet. 11: 636-646 (2010), which is hereby incorporated by reference in its entirety). By taking advantage of endogenous DNA repair machinery, these reagents can be used to precisely alter the genomes of higher organisms. The use of ZFNs to target long non-coding RNA has been described in the art (Gutschner et al., “Noncoding RNA Gene Silencing Through Genomic Integration of RNA Destabilizing Elements Using Zinc Finger Nucleases,” Genome Res. 21:1944-1954 (2011), which is hereby incorporated by reference in its entirety).

In another embodiment, the therapeutic agent for inhibiting NOTCH-1 regulated lncRNA expression is a meganuclease and TAL effector nuclease (TALENs, Cellectis Bioresearch) (Joung & Sander, “TALENs: A Widely Applicable Technology for Targeted Genome Editing,” Nat. Rev. Mol. Cell Biol. 14: 49-55 (2013), which is hereby incorporated by reference in its entirety). A TALEN® is composed of a TALE DNA binding domain for sequence-specific recognition fused to the catalytic domain of an endonuclease that introduces double strand breaks (DSB). The DNA binding domain of a TALEN® is capable of targeting with high precision a large recognition site (for instance 17 bp). Meganucleases are sequence-specific endonucleases, naturally occurring “DNA scissors”, originating from a variety of single-celled organisms such as bacteria, yeast, algae and some plant organelles. Meganucleases have long recognition sites of between 12 and 30 base pairs. The recognition site of natural meganucleases can be modified in order to target native genomic DNA sequences (such as endogenous genes).

In another embodiment, the therapeutic agent for inhibiting NOTCH-1 regulated lncRNA expression is a CRISPR-Cas9 guided nuclease (Wiedenheft et al., “RNA-Guided Genetic Silencing Systems in Bacteria and Archaea,” Nature 482:331-338 (2012); Zhang et al., “Multiplex Genome Engineering Using CRISPR/Cas Systems,” Science 339(6121): 819-23 (2013); and Gaj et al., “ZFN, TALEN, and CRISPR/Cas-based Methods for Genome Engineering,” Cell 31(7):397-405 (2013), which are hereby incorporated by reference in their entirety). Like the TALENs and ZFNs, CRISPR-Cas9 interference is a genetic technique which allows for sequence-specific control of gene expression in prokaryotic and eukaryotic cells by guided nuclease double-stranded DNA cleavage. It is based on the bacterial immune system-derived CRISPR (clustered regularly interspaced palindromic repeats) pathway. The use of CRISPR-Cas9 technology to target long non-coding RNA has been described in the art (Ho et al., “Targeting Non-coding RNAs With the CRISPR/Cas9 System in Human Cell Lines,” Nucl. Acids. Res. 43(3):e17 (2014); “Han et al., “Efficient In Vivo Deletion of a Large Imprinted lncRNA by CRISPR/Cas9,” RNA Biol. 11(7):829-835 (2014), which are hereby incorporated by reference in their entirety).

Inhibition of NOTCH-1 regulated lncRNAs can also be carried out using antisense oligonucleotides (ASO). The use of ASO technology to target long non-coding RNA has been described in the art (Zhou et al., “Targeting Long Noncoding RNA with Antisense Oligonucleotide Technology as Cancer Therapeutics,” Methods Mol. Biol. 1402:199-213 (2016), which is hereby incorporated by reference in its entirety). Accordingly, suitable therapeutic ASOs for inhibition of lncRNAs such as LUNAR1, Linc94, or Lin_CXCR4 include, without limitation, antisense RNAs, DNAs, RNA/DNA hybrids (e.g., gapmer), and chemical analogues thereof, e.g., morpholinos, peptide nucleic acid oligomer, ASOs comprised of locked nucleic acids. With the exception of RNA oligomers, PNAs, and morpholinos, all other antisense oligomers act in eukaryotic cells through the mechanism of RNase H-mediated target cleavage. PNAs and morpholinos bind complementary DNA and RNA targets with high affinity and specificity, and thus act through a simple steric blockade of the RNA translational machinery, and appear to be completely resistant to nuclease attack.

An “antisense oligomer” refers to an antisense molecule or anti-gene agent that comprises an oligomer of at least about 10 nucleotides in length. In embodiments an antisense oligomer comprises at least 15, 18, 20, 25, 30, 35, 40, or 50 nucleotides. Antisense approaches involve the design of oligonucleotides (either DNA, RNA, DNA/RNA, or chemically modified derivatives thereof) that are complementary to an RNA encoded by polynucleotide sequences of the NOTCH1-regulated lncRNAs identified herein, e.g., LUNAR1. Antisense RNA may be introduced into a cell to inhibit translation or activity of a complementary mRNA by base pairing to it and physically obstructing its translation or its activity. This effect is therefore stoichiometric. Absolute complementarity, although preferred, is not required. A sequence “complementary” to a portion of an RNA, as referred to herein, means a sequence having sufficient complementarity to be able to hybridize with the RNA, forming a stable duplex. In the case of double stranded antisense polynucleotide sequences, a single strand of the duplex DNA may thus be tested, or triplex formation may be assayed. The ability to hybridize will depend on both the degree of complementarity and the length of the antisense polynucleotide sequence. Generally, the longer the hybridizing polynucleotide sequence, the more base mismatches with an RNA it may contain and still form a stable duplex (or triplex, as the case may be). One skilled in the art can ascertain a tolerable degree of mismatch by use of standard procedures to determine the melting point of the hybridized complex.

In one embodiment, the therapeutic agent is an antisense oligonucleotide that specifically binds to and inhibits the functional expression of LUNAR1. Exemplary LUNAR1 ASOs are provided herein and have the nucleotide sequence of SEQ ID NO: 71 or 72. The ASOs of SEQ ID NOs: 71 and 72 can be readily modified using techniques known in the art to increase specific and stable hybridization of the ASO to LUNAR1 lncRNA as well as to increase resistance of the ASO to degradation by nucleases present within the body (see Chan et al., “Antisense Oligonucleotides: From Design to Therapeutic Application,” Clin. Exp. Pharm. Physiol. 33: 533-540 (2006), which is hereby incorporated by reference in its entirety). For example, common modifications to the ASO to increase duplex stability include the incorporation of 5-methyl-dC, 2-amino-dA, locked nucleic acid, and/or peptide nucleic acid bases. Common modifications to enhance nuclease resistance include conversion of the normal phosphodiester linkages to phosphorothioate or phosphorodithioate linkages, or use of propyne analog bases, 2′-O-Methyl or 2′-O-Methyloxyethyl RNA bases. Exemplary modified ASO inhibitors of LUNAR1 are provided herein as SEQ ID NOs: 47 and 48. These modified ASOs, which are derived from the nucleotide sequences of SEQ ID NOs: 71 and 72, respectively, contain phosphorothioate bonds and 2′-O-Methyl RNA bases at the indicated bases. In addition to the ASOs having nucleotide sequences of SEQ ID NOs: 71 and 72, other LUNAR1 ASOs having different nucleotide sequences, and derivatives there of, can readily be designed and tested based on the sequence of LUNAR1 provided herein and are contemplated and within the scope of the present invention.

In one embodiment, the therapeutic agent is an antisense oligonucleotide that specifically binds to and inhibits the functional expression of Lin_CXCR4. Exemplary Lin_CXCR4 ASOs are provided herein and have the nucleotide sequence of SEQ ID NO: 73 or 74, respectively. As noted above, the ASOs of SEQ ID NOs: 73 and 74 can be modified to increase duplex stability (e.g., methyl-dC, 2-amino-dA, and/or locked nucleic acid bases) and increase resistance to degradation (e.g., phosphorothioate or phosphorodithioate linkages, propyne analog bases, or 2′O-Methyl RNA bases). Exemplary modified ASO inhibitors of Lin_CXCR4 are provided as SEQ ID NOs: 49 and 50. These modified ASOs, which are derived from the nucleotide sequences of SEQ ID NOs: 73 and 74, respectively, contain phosphorothioate bonds and 2′O-methyl RNA bases at the indicated bases. In addition to the ASOs having the nucleotide sequence of SEQ ID NOs: 73 and 74, other Lin_CXCR4 ASOs having different nucleotide sequences, and derivatives thereof, can readily be designed and tested based on the sequence of Lin_CXCR4 provided herein and are contemplated and within the scope of the present invention.

RNA interference (RNAi) using small interfering RNA (siRNA) is another form of post-transcriptional gene silencing that can be utilized for inhibiting NOTCH-1 regulated lncRNAs in a subject as described herein.

Accordingly, in one embodiment, the therapeutic agent that inhibits a NOTCH-1 regulated lncRNA is an siRNA. siRNAs are double stranded synthetic RNA molecules approximately 20-25 nucleotides in length with short 2-3 nucleotide 3′ overhangs on both ends. The double stranded siRNA molecule represents the sense and anti-sense strand of a portion of the target mRNA molecule, in this case a portion of a NOTCH-1 regulated lncRNA (e.g., LUNAR1, Lin_CXCR4, Linc94, or any of the lnRNAs of Table 1). siRNA molecules are typically designed to target a region of the mRNA target approximately 50-100 nucleotides downstream from the start codon. The siRNAs of the invention can comprise partially purified RNA, substantially pure RNA, synthetic RNA, or recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siRNA or to one or more internal nucleotides of the siRNA, including modifications that make the siRNA resistant to nuclease digestion. Upon introduction into a cell, the siRNA complex triggers the endogenous RNAi pathway, resulting in the cleavage and degradation of the target mRNA molecule. Various improvements of siRNA compositions, such as the incorporation of modified nucleosides or motifs into one or both strands of the siRNA molecule to enhance stability, specificity, and efficacy, have been described and are suitable for use in accordance with this aspect of the invention (see e.g., WO2004/015107 to Giese et al.; WO2003/070918 to McSwiggen et al.; WO1998/39352 to Imanishi et al.; U.S. Patent Application Publication No. 2002/0068708 to Jesper et al.; U.S. Patent Application Publication No. 2002/0147332 to Kaneko et al; U.S. Patent Application Publication No. 2008/0119427 to Bhat et al., which are hereby incorporated by reference in their entirety).

In another embodiment, the therapeutic agent comprises endoribonuclease-prepared siRNAs (esiRNA), which comprise a mixture of siRNA oligonucleotides formed from the cleavage of long double stranded RNA with an endoribonuclease (e.g., RNase III or dicer). Digestion of synthetic long double stranded RNA produces short overlapping fragments of siRNAs with a length of between 18-25 bases that all target the same mRNA sequence. The complex mixture of many different siRNAs all targeting the same mRNA sequence leads to increased silencing efficacy. The use of esiRNA technology to target long non-coding RNA has been described in the art (Theis et al., “Targeting Human Long Noncoding Transcripts by Endoribonuclease-Prepared siRNAs,” J. Biomol. Screen 20(8):1018-1026 (2015), which is hereby incorporated by reference in its entirety).

In one embodiment, the therapeutic agent is a short or small hairpin RNA. Short or small hairpin RNA molecules are similar to siRNA molecules in function, but comprise longer RNA sequences that make a tight hairpin turn. shRNA is cleaved by cellular machinery into siRNA and gene expression is silenced via the cellular RNA interference pathway.

In one embodiment, the therapeutic agent is a shRNA that inhibits the functional expression of LUNAR1. Exemplary LUNAR1 shRNAs are provided herein, having the nucleotide sequences of SEQ ID NOs: 41 and 42, respectively. However, other LUNAR1 shRNAs, which can readily be designed and tested based on the sequence of LUNAR1 provided herein, are also contemplated and within the scope of the present invention.

In one embodiment, the therapeutic agent is a shRNA that inhibits the functional expression of Linc94. An exemplary Linc94 shRNA is provided herein, having the nucleotide sequences of SEQ ID NO: 45. However, other Linc94 shRNAs, which can readily be designed and tested based on the sequence of Linc94 provided herein, are also contemplated and within the scope of the present invention.

Nucleic acid aptamers that specifically bind to NOTCH-1 regulated lncRNAs are also useful in the methods of the present invention. Nucleic acid aptamers are single-stranded, partially single-stranded, partially double-stranded, or double-stranded nucleotide sequences, advantageously a replicatable nucleotide sequence, capable of specifically recognizing a selected non-oligonucleotide molecule or group of molecules by a mechanism other than Watson-Crick base pairing or triplex formation. Aptamers include, without limitation, defined sequence segments and sequences comprising nucleotides, ribonucleotides, deoxyribonucleotides, nucleotide analogs, modified nucleotides, and nucleotides comprising backbone modifications, branchpoints, and non-nucleotide residues, groups, or bridges. Nucleic acid aptamers include partially and fully single-stranded and double-stranded nucleotide molecules and sequences; synthetic RNA, DNA, and chimeric nucleotides; hybrids; duplexes; heteroduplexes; and any ribonucleotide, deoxyribonucleotide, or chimeric counterpart thereof and/or corresponding complementary sequence, promoter, or primer-annealing sequence needed to amplify, transcribe, or replicate all or part of the aptamer molecule or sequence.

The NOTCH-1 regulated lncRNA inhibitors of the present invention are packaged in a suitable delivery vehicle or carrier for delivery to the subject. Suitable delivery vehicles include, but are not limited to viruses, virus-like particles, bacteria, bacteriophages, biodegradable microspheres, microparticles, nanoparticles, exosomes, liposomes, collagen minipellets, and cochleates. These and other biological gene delivery vehicles are well known to those of skill in the art (see e.g., Seow and Wood, “Biological Gene Delivery Vehicles: Beyond Viral Vectors,”Mol. Therapy 17(5):767-777(2009), which is hereby incorporated by reference in its entirety).

In one embodiment, the therapeutic inhibitor of NOTCH-1 regulated lncRNA is packaged into a therapeutic expression vector to facilitate delivery. Suitable expression vectors are well known in the art and include, without limitation, viral vectors such as adenovirus vectors, adeno-associated virus vectors, retrovirus vectors, lentivirus vectors, or herpes virus vectors. The viral vectors or other suitable expression vectors comprise sequences encoding the inhibitory nucleic acid molecule (e.g., siRNA, ASO, etc.) of the invention and any suitable promoter for expressing the inhibitory sequences. Suitable promoters include, for example, and without limitation, the U6 or HI RNA pol III promoter sequences and the cytomegalovirus promoter. Selection of other suitable promoters is within the skill in the art. The expression vectors may also comprise inducible or regulatable promoters for expression of the inhibitory nucleic acid molecules in a tissue or cell-specific manner.

Gene therapy vectors carrying the therapeutic inhibitory nucleic acid molecule are administered to a subject by, for example, intravenous injection, local administration (U.S. Pat. No. 5,328,470 to Nabel et al., which is hereby incorporated by reference in its entirety) or by stereotactic injection (see e.g., Chen et al. “Gene Therapy for Brain Tumors: Regression of Experimental Gliomas by Adenovirus Mediated Gene Transfer In Vivo,” Proc. Nat'l. Acad. Sci. USA 91:3054-3057 (1994), which is hereby incorporated by reference in its entirety). The pharmaceutical preparation of the therapeutic vector can include the therapeutic vector in an acceptable diluent, or can comprise a slow release matrix in which the therapeutic delivery vehicle is imbedded. Alternatively, where the complete therapeutic delivery vector can be produced intact from recombinant cells, e.g., retroviral vectors, the pharmaceutical preparation can include one or more cells which produce the therapeutic delivery system. Gene therapy vectors typically utilize constitutive regulatory elements which are responsive to endogenous transcriptions factors.

Another suitable approach for the delivery of the therapeutic agents of the present invention, involves the use of liposome delivery vehicles or nanoparticle delivery vehicles.

In one embodiment of the present invention, the pharmaceutical composition or formulation containing an inhibitory nucleic acid molecule (e.g., siRNA molecule) is encapsulated in a lipid formulation to form a nucleic acid-lipid particle as described in Semple et al., “Rational Design of Cationic Lipids for siRNA Delivery,” Nature Biotech. 28:172-176 (2010) and International Patent Application Publication Nos. WO2011/034798 to Bumcrot et al., WO2009/111658 to Bumcrot et al., and WO2010/105209 to Bumcrot et al., which are hereby incorporated by reference in their entirety. Other cationic lipid carriers suitable for the delivery of ASO include, without limitation, N-[1-(2,3-dioleoyloxy)propyl]-N,N,N-trimethylammonium chloride (DOTMA) and N-[1-(2,3-dioleoyloxy)propyl]-N,N,N-trimethylammonium methyl sulphate (DOTAP) (see Chan et al., “Antisense Oligonucleotides: From Design to Therapeutic Application,” Clin. Exp. Pharm. Physiol. 33: 533-540 (2006), which is hereby incorporated by reference in its entirety).

In another embodiment of the present invention, the delivery vehicle is a nanoparticle. A variety of nanoparticle delivery vehicles are known in the art and are suitable for delivery of therapeutic agent of the invention (see e.g., van Vlerken et al., “Multi-functional Polymeric Nanoparticles for Tumour-Targeted Drug Delivery,” Expert Opin. Drug Deliv. 3(2):205-216 (2006), which is hereby incorporated by reference in its entirety). Suitable nanoparticles include, without limitation, poly(beta-amino esters) (Sawicki et al., “Nanoparticle Delivery of Suicide DNA for Epithelial Ovarian Cancer Cell Therapy,” Adv. Exp. Med. Biol. 622:209-219 (2008), which is hereby incorporated by reference in its entirety), polyethylenimine-alt-poly(ethylene glycol) copolymers (Park et al., “Degradable Polyethylenimine-alt-Poly(ethylene glycol) Copolymers As Novel Gene Carriers,” J. Control Release 105(3):367-80 (2005) and Park et al., “Intratumoral Administration of Anti-KITENIN shRNA-Loaded PEI-alt-PEG Nanoparticles Suppressed Colon Carcinoma Established Subcutaneously in Mice,” J Nanosci. Nanotechnology 10(5):3280-3 (2010), which are hereby incorporated by reference in their entirety), poly(d,l-lactide-coglycolide) (Chan et al., “Antisense Oligonucleotides: From Design to Therapeutic Application,” Clin. Exp. Pharm. Physiol. 33: 533-540 (2006), which is hereby incorporated by reference in its entirety), and liposome-entrapped siRNA nanoparticles (Kenny et al., “Novel Multifunctional Nanoparticle Mediates siRNA Tumor Delivery, Visualization and Therapeutic Tumor Reduction In Vivo,” J. Control Release 149(2): 111-116 (2011), which is hereby incorporated by reference in its entirety). Other nanoparticle delivery vehicles suitable for use in the present invention include microcapsule nanotube devices disclosed in U.S. Patent Publication No. 2010/0215724 to Prakash et al., which is hereby incorporated by reference in its entirety.

In another embodiment of the present invention, the pharmaceutical composition is contained in a liposome delivery vehicle. The term “liposome” means a vesicle composed of amphiphilic lipids arranged in a spherical bilayer or bilayers. Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo.

Several advantages of liposomes include: their biocompatibility and biodegradability, incorporation of a wide range of water and lipid soluble drugs; and they afford protection to encapsulated drugs from metabolism and degradation. Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes and as the merging of the liposome and cell progresses, the liposomal contents are emptied into the cell where the active agent may act.

Methods for preparing liposomes for use in the present invention include those disclosed in Bangham et al., “Diffusion of Univalent Ions Across the Lamellae of Swollen Phospholipids,” J. Mol. Biol. 13:238-52 (1965); U.S. Pat. No. 5,653,996 to Hsu; U.S. Pat. No. 5,643,599 to Lee et al.; U.S. Pat. No. 5,885,613 to Holland et al.; U.S. Pat. No. 5,631,237 to Dzau & Kaneda; and U.S. Pat. No. 5,059,421 to Loughrey et al., which are hereby incorporated by reference in their entirety.

The liposome and nanoparticle delivery systems can be made to accumulate at a target organ, tissue, or cell via active targeting (e.g., by incorporating an antibody or other ligand on the surface of the delivery vehicle). For example, when targeting T-cell acute lymphoblastic leukemia, as in the present invention, the delivery vehicle may be conjugated to an anti-TALLA-1 antibody as disclosed by Takagi et al., “Identification of a Highly Specific Surface Marker of T-cell Acute Lymphoblastic Leukemia and Neuroblastoma as a New Member of the Transmembrane 4 Superfamily,” Int. J. Cancer 61(5):706-15 (1995), which is hereby incorporated by reference in its entirety, or other antibody recognizing T-ALL specific surface markers.

As used herein, an “effective amount” of the inhibitory agent, such as an siRNA, shRNA, or the like, is an amount sufficient to cause inhibition or degradation of the target lncRNA. RNAi or ASO mediated inhibition of the target lncRNA can be detected by measuring levels of the target lncRNA in the cells of a subject, using standard techniques for isolating and quantifying mRNA as described herein. One skilled in the art can readily determine an effective amount of the therapeutic agent of the invention to be administered to a given subject, by taking into account factors such as the size and weight of the subject; the extent or stage of the leukemia; the age, health and sex of the subject; the route of administration; and whether the administration is regional or systemic. Generally, an effective amount of the inhibitory agent of the invention, e.g., siRNA or ASO, has an intracellular concentration of from about 1 nanomolar (nM) to about 100 nM, preferably from about 2 nM to about 50 nM, more preferably from about 2.5 nM to about 10 nM. It is contemplated that greater or lesser amounts of the therapeutic inhibitory agent can be administered.

In some embodiments of the present invention, the inhibitor of a NOTCH-1 regulated lncRNA is administered in combination with another T-ALL therapeutic agent. The current standard therapy for T-ALL includes the administration of one or more chemotherapeutic reagents. Accordingly, the NOTCH-1 regulated lncRNA inhibitor can be administered to a subject prior to, concurrent with, or following the administration of a chemotherapeutic reagent. In accordance with this aspect of the present invention, the chemotherapeutic agent is selected from the group consisting of cytarabine, vincristine, prednisone, doxorubicin, daunorubicin, PEG asparaginase, methotrexate, cyclophosphamide, L-asparaginase, etoposide, and leucovorin.

In a further embodiment, the therapeutic agent that inhibits a NOTCH-1 regulated long non-coding RNA is administered in combination with a Notch-1 antagonist. Because activation of the Notch-1 signaling pathway has been observed in over 80% of all T-cell acute lymphoblastic leukemia cases, a number of Notch-1 antagonists have been developed and are well known in the art. Suitable Notch-1 antagonists include without limitation gamma-secretase inhibitors selected from the group consisting of [(25)-2-{[(3,5-Difluorophenyeacetyl]amino}-N-[3S)1-methyl-2-oxo-5-phenyl-2,3-dihydro-1H-1,4-benzodiazepin-3-yl]propanamide] (CompE), N4N-(3,5-difluorophenacetyl)-L-alanyl]-Sphenylglycine-t-butylester (DAPT), LY411575, (5S)-(t-Butoxycarbonylamino)-6-phenyl-(4R)hydroxy-(2R)benzylhexanoyl)-L-leu-L-phe-amide (L-685,458), L-852,647, MW167, WPE-111-31, LY450139, MRK003, R-flurbiprofen ([1,1′-Biphenyl]-4-acetic acid, 2-fluoro-alpha-methyl), NGX-555, CZC-1040, E2012, GSI-1, Begacestat (2-Thiophenesulfonamide, 5-chloro-N-[(1S)-3₁3,3-trifluoro-1-(hydroxymethyl)-2-(trifluoromethyl)propyl]-), NIC5-15, BACE Inhibitor, and CHF-5074.

In practicing the methods of the present invention, the administering step is carried out to achieve inhibition of NOTCH-1 regulated lncRNAs in T-ALL cells. Such administration can be carried out systemically or via direct or local administration. By way of example, suitable modes of systemic administration include, without limitation orally, topically, transdermally, parenterally, intradermally, intramuscularly, intraperitoneally, intravenously, subcutaneously, or by intranasal instillation, by intracavitary or intravesical instillation, intraocularly, intraarterialy, intralesionally, or by application to mucous membranes. Suitable modes of local administration include, without limitation, catheterization, implantation, direct injection, dermal/transdermal application, or portal vein administration to relevant tissues, or by any other local administration technique, method or procedure generally known in the art.

Therapeutic agents of the present invention are formulated in accordance with their mode of administration. For oral administration, for example, the therapeutic agents of the present invention are formulated into an inert diluent or an assimilable edible carrier and enclosed in biodegradable particle (Akhtar S., “Oral Delivery of siRNA and Antisense Oligonucleotides,” J. Drug Targeting 17(7): 491-95 (2009), which is hereby incorporated by reference in its entirety). Agents of the present invention may also be administered in a time release manner incorporated within such devices as time-release capsules or nanotubes. Such devices afford flexibility relative to time and dosage. For oral therapeutic administration, the agents of the present invention may be incorporated with excipients and used in the form of tablets, capsules, elixirs, suspensions, syrups, and the like. Such compositions and preparations should contain at least 0.1% of the agent, although lower concentrations may be effective and indeed optimal. The percentage of the agent in these compositions may, of course, be varied and may conveniently be between about 2% to about 60% of the weight of the unit. The amount of an agent of the present invention in such therapeutically useful compositions is such that a suitable dosage will be obtained.

The therapeutic agents of the present invention may also be formulated for parenteral administration. Solutions or suspensions of the agent can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

Pharmaceutical formulations suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

When it is desirable to deliver the agents of the present invention systemically, they may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

In addition to the formulations described previously, the agents may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

Effective doses of the therapeutic agents of the present invention vary depending upon many different factors, including type and stage of leukemia, mode of administration, target site, physiological state of the patient, other medications or therapies administered, and physical state of the patient relative to other medical complications. Treatment dosages need to be titrated to optimize safety and efficacy. Effective doses can be achieved and maintained by repeat administration, i.e., hourly, daily, weekly, monthly, yearly administration of the therapeutic lncRNA inhibitor.

Another aspect of the present invention is directed to a nucleic acid construct capable of silencing or inhibiting LUNAR1 expression and/or activity. Suitable inhibitors include any and all of those described above. In one embodiment, the inhibitor of LUNAR1 is an antisense oligonucleotide, i.e., a single-stranded oligonucleotide having a nucleobase sequence that permits hybridization to a corresponding region or segment of the LUNAR1 nucleotide sequence. Exemplary LUNAR1 ASOs are described herein, e.g., ASOs having the nucleotide sequences of SEQ ID NOs: 71 and 72 and derivatives thereof, such as the modified ASOs of SEQ ID NOs: 47 and 48, respectively. In another embodiment the LUNAR1 inhibitor is an RNAi, e.g., an siRNA or shRNA. Exemplary LUNAR1 shRNAs are described herein, e.g., shLUNAR1 having the nucleotide sequences of SEQ ID NOs: 41 and 42.

Another aspect of the present invention is directed to an isolated cDNA construct encoding LUNAR1. An exemplary isolated LUNAR1 cDNA is disclosed herein having a nucleotide sequence of SEQ ID NO: 1. In one embodiment, the isolated LUNAR cDNA is the full length nucleotide sequence of SEQ ID NO: 1. In another embodiment, the isolated LUNAR1 cDNA is a fragment of the nucleotide sequence of SEQ ID NO: 1. In another embodiment, the isolated LUNAR1 cDNA has one or more insertion, deletions, or nucleotide substitutions relative to the nucleotide sequence of SEQ ID NO: 1. Preferably, the nucleotide sequence of the isolated LUNAR1 cDNA of the present invention has at least about 70%, 75%, 80%, 85%, 90%, 95%, 96%, 97%, 98% or 99% sequence similarity to the nucleotide sequence of SEQ ID NO: 1.

Another aspect of the present invention is directed to a nucleic acid construct capable of silencing or inhibiting Lin_CXCR4 expression and/or activity. Suitable inhibitors include any and all of those described above. In one embodiment, the inhibitor of Lin_CXCR4 is an antisense oligonucleotide, i.e., a single-stranded oligonucleotide having a nucleobase sequence that permits hybridization to a corresponding region or segment of the Lin_CXCR4 nucleotide sequence. Exemplary Lin_CXCR4 ASOs are described herein, e.g., ASOs having the nucleotide sequences of SEQ ID NOs: 73 and 74 and derivatives thereof, such as the modified ASOs of SEQ ID NOs: 49 and 50, respectively. In another embodiment the Lin_CXCR4 inhibitor is an RNAi, e.g., an siRNA or shRNA.

Another aspect of the present invention relates to a method of diagnosing T cell acute lymphoblastic leukemia in a subject. This method involves measuring, in a biological sample obtained from a subject, expression levels of one or more NOTCH-1 regulated lncRNA transcripts, and comparing the measured expression levels to control expression levels of the one or more NOTCH-1 regulated lncRNA transcripts. The subject is then diagnosed as having T cell acute lymphoblastic leukemia based on the comparing.

In accordance with this aspect of the invention the NOTCH-1 regulated lncRNA is any one or more of the 130 lncRNAs listed in Table 1 above. In one embodiment of the present invention, the expression levels of at least 2, 3, 4, 5, 6, 7, 8, 9, 10, 15, or 20 of the lncRNAs listed in Table 1 are measured for ascertaining a T-ALL diagnosis. In accordance with this aspect of the invention, an increase in the expression of the one or more NOTCH-1 regulated lncRNAs in the biological sample from the subject relative to control expression levels indicates a positive diagnosis of T-ALL in the subject.

In one embodiment, T-ALL diagnosis is determined based on the expression level of at least LUNAR 1. In another embodiment, T-ALL diagnosis is determined based on the expression level of at least lnc_CXCR4. In another embodiment, T-ALL diagnosis is determined based on the expression level of at least Linc94. In another embodiment, T-ALL diagnosis is determined based on the expression levels of any combination of LUNAR1, lnc-CXCR4, and/or Linc94.

The biological sample can be any biological fluid, tissue, or cell obtained or otherwise derived from a subject including, but not limited to, blood (including whole blood, leukocytes, peripheral blood mononuclear cells, plasma, and serum), sputum, mucus, nasal aspirate, urine, semen, saliva, meningeal fluid, lymph fluid, milk, bronchial aspirate, a cellular extract, and cerebrospinal fluid. This also includes experimentally separated fractions of all of the preceding. For example, a blood sample can be fractionated into serum or into fractions containing particular types of blood cells, such as red blood cells or white blood cells (leukocytes). If desired, a sample may be a combination of samples from an individual, such as a combination of a tissue and fluid sample. A biological sample may also include materials containing homogenized solid material, such as a tissue sample, or a tissue biopsy; or materials derived from a tissue culture or a cell culture.

In one embodiment, the biological sample comprises leukemia-initiating cells. As described herein, leukemia initiating cells represent a subset of leukemic cells that possess properties similar to normal hematopoietic stem cells such as self-renewal, quiescence, and resistance to traditional chemotherapy (Bonnet & Dick, “Human Acute Myeloid Leukemia is Organized as a Hierarchy That Originates From a Primitive Hematopoietic Cell,” Nat. Med. 3:730-737 (1997); Huntly & Gilliland, “Leukaemia Stem Cells and the Evolution of Cancer-Stem-Cell Research,” Nat. Rev. Cancer 5:311-321 (2005), which are hereby incorporated by reference in their entirety). As a result, the LIC subset acts as a reservoir of cells contributing to disease, in particular disease relapse. LIC populations have been identified in acute myeloid leukemia, chronic phase and blast crisis, T-cell acute lymphoblastic leukemia, and B-cell acute lymphoblastic leukemia.

The methods of the present invention are suitable for diagnosing and distinguishing all subtypes and stages of T-ALL including, but not limited to, adult T-cell acute lymphoblastic leukemia, pediatric T-cell acute lymphoblastic leukemia, early T-cell precursor acute lymphoblastic leukemia, NOTCH-1 mediated T-ALL, relapsed T-cell acute lymphoblastic leukemia, and refractory T-cell acute lymphoblastic leukemia.

The diagnostic methods described herein can be used to guide therapeutic treatment. For example, the detection of one more lncRNAs as described herein (e.g., LUNAR1, lnc_CXCR4, and/or Linc94), is diagnostic of NOTCH-1 mediated T-ALL and warrants consideration of including a Notch inhibitor in the therapeutic regimen. Accordingly, in one embodiment, this method of the invention further involves administering a therapeutic agent based on the diagnosis.

In accordance with this aspect of the present invention, the expression levels of one or more lncRNA transcripts in a sample are measured, quantified and/or detected by any suitable RNA detection, quantification or sequencing methods known in the art, including, but not limited to, reverse transcriptase-polymerase chain reaction (RT-PCR) methods, northern blot, microarray, serial analysis of gene expression (SAGE), next-generation RNA sequencing (e.g., deep sequencing, whole transcriptome sequencing, exome sequencing), gene expression analysis by massively parallel signature sequencing (MPSS), immune-derived colorimetric assays, in situ hybridization (ISH) formulations (colorimetric/radiometric) that allow histopathology analysis, mass spectrometry (MS) methods, and RNA pull-down and chromatin isolation by RNA purification (ChiRP). In one embodiment, the method of measuring an lncRNA transcript (or modification thereof) level includes performing quantitative/gel-based electrophoresis PCR or non-PCR-based molecular amplification methods for detection.

The expression levels of the one or more lncRNA transcripts from the biological sample are compared to “control” expression levels of the same one or more lncRNA transcripts to provide a diagnosis. The control expression levels of the one or more lncRNA transcripts can be the average expression level of the one or more lncRNA transcripts in biological samples taken from healthy individuals. In another embodiment, the control expression levels can be derived from the expression levels of the one or more lncRNA transcripts in a biological sample taken from the same subject being diagnosed but at an earlier timepoint. The measured expression levels can be either in absolute amount (e.g., number of copies/ml, nanogram/ml or microgram/ml) or a relative amount (e.g., relative intensity of signals).

Another aspect of the present invention is directed to a kit suitable for diagnosing T-ALL. This kit includes one or more reagents suitable for detecting the expression levels of one or more NOTCH-1 regulated lncRNAs as described herein using any of the detection/quantification methods described supra. In accordance with this aspect of the present invention, the kit comprises one or more reagents suitable for detecting the expression levels of the one or more NOTCH-1 regulated lncRNAs listed in Table 1. In one embodiment, the kit contains reagents to detect at least two lncRNAs, at least three lncRNAs, at least four lncRNAs, at least five lncRNAs, or at least 6, 7, 8,9, 10, 15, 20, 25, 30, 35, 40, 45, 50, 55, 60, 65, 70, 75, 80, 85, 90, 95, 100, 105, 110, 115, 120, 125, or all 130 of the lncRNA listed in Table 1.

A number of kits are contemplated to encompass a variety of methods. These kits optionally include reagents to process a tissue or cell sample for the technique employed by that particular kit. By example, a kit for PCR or PCR enhanced in situ hybridization can include reagents to process the sample and isolate the RNA (for PCR). It will also contain suitable primers to amplify the target sequence and additional probes, if necessary, to detect the desired nucleic acid fragments as well as buffers and reagents for the polymerase chain reaction and the buffers and emulsions required for in situ hybridization methods. Other kits can alternatively include probes, primers and reagents for modifications of in situ or PCR in situ hybridization methods.

In one embodiment, the kit includes oligonucleotide primers or probes suitable for detecting the expression of LUNAR1. In another embodiment, the kit includes oligonucleotide primers or probes suitable for detecting the expression of lnc_CXCR4. In another embodiment, the kit includes oligonucleotide primers or probes suitable for detecting the expression of Linc94. In yet another embodiment, the kit includes oligonucleotide primers or probes suitable for detecting any combination of LUNAR1, lnc_CXCR4, and/or Linc94.

In one embodiment, the kit includes oligonucleotide primers suitable for use in a quantitative PCR reaction. Exemplary qPCR primers for the detection of LUNAR1 include, but are not limited to those shown in Table 2 infra, e.g., SEQ ID NOs: 14 and 15 or 22 and 23. Exemplary qPCR primers for the detection of lnc_CXCR4 include, but are not limited to those shown in Table 2 infra, e.g., SEQ ID NOs: 26 and 27. Exemplary qPCR primers for the detection of Linc94 include, but are not limited to those shown in Table 2 infra, e.g., SEQ ID NOs: 18 and 19. Additional primers suitable qPCR detection can readily be designed based on the LUNAR1, lnc_CXC4, and Linc94 nucleotide sequence disclosed supra.

In another embodiment, the kit includes oligonucleotide probes suitable for carrying out ChIRP assay as described herein. Exemplary LUNAR1 ChIRP probes are disclosed in Table 2, infra, e.g., SEQ ID NOs: 51-60. Additional LUNAR1 probes suitable ChIRP can readily be designed based on the LUNAR1 nucleotide sequence disclosed supra.

### EXAMPLES

The examples below are intended to exemplify the practice of the present invention but are by no means intended to limit the scope thereof.

### Materials and Methods for Examples 1-6

Primary T-ALL Samples. Primary T-ALL samples were provided by the Children's Oncology Group with informed consent and analyzed under the supervision of the New York University Langone Medical Center Institutional Review Board.

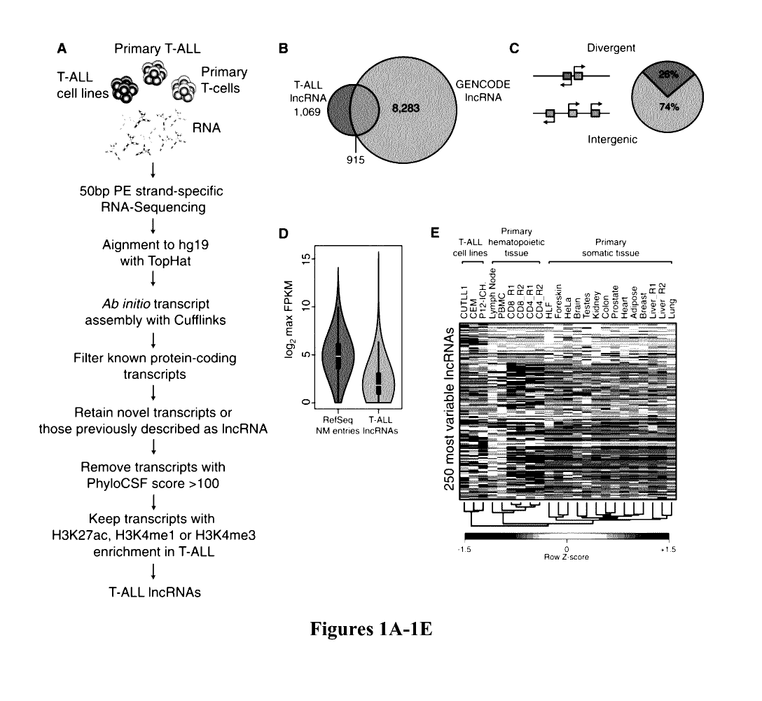

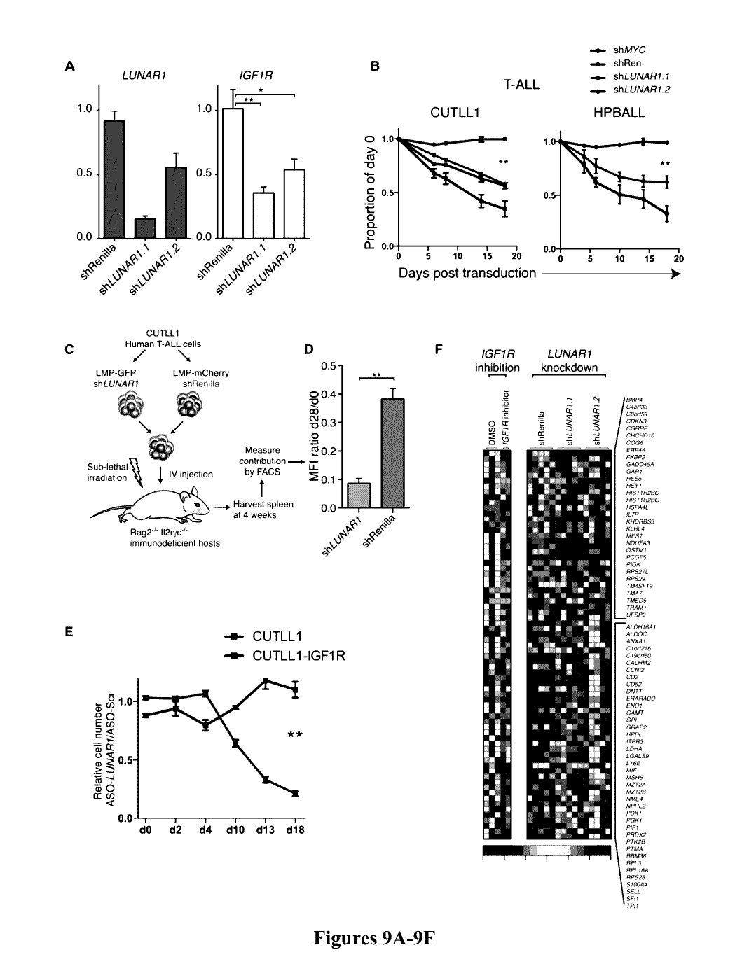

RNA Extraction Preparation for Next-Generation Sequencing. Total RNA was extracted from samples using the RNeasy Plus mini kit (Life Technologies, Carlsbad, Calif., USA). Samples were then subject to poly(A) selection (FIGS. 1E, 9F, and 9G only) using oligo-dT beads (Life Technologies) or rRNA removal (all other samples) using the Ribo-Zero kit (Epicenter, Madison, Wis., USA), according to the manufacturer's instructions. The resulting RNA samples were then used as input for library construction using the dUTP method as described (Parkhomchuk et al., “Transcriptome Analysis by Strand-specific Sequencing of Complementary DNA,” Nucleic Acids Res. 37:e123 (2009), which is hereby incorporated by reference in its entirety). RNA libraries were then sequenced on the Illumina HiSeq 2000 or 2500 using 50 bp paired-end reads.

TABLE 2
Primer Sequences, shRNA Sequences, and ASO Sequences

| Name | Sequence |
|---|---|
| qPCR cDNA primers | |
| hGAPDH_F | CTTTTGCGTCGCCAGCCGAG (SEQ ID NO: 4) |
| hGAPDH_R | CCAGGCGCCCAATACGACCA (SEQ ID NO: 5) |
| hIGF1R_F | TACTCGGACGTCTGGTCCTT (SEQ ID NO: 6) |
| hIGF1R_R | TGGGGTTATACTGCCAGCAC (SEQ ID NO: 7) |
| hDTX1_F | CAGCTTGTGCCCTACATCATC (SEQ ID NO: 8) |
| hDTX1_R | ACGACGGGTCGTAGAAGTTG (SEQ ID NO: 9) |
| hHES1_F | GCAGATGACGGCTGCGCTGA (SEQ ID NO: 10) |
| hHES1_R | AAGCGGGTCACCTCGTTCATGC (SEQ ID NO: 11) |
| hU1_F | ATACTTACCTGGCAGGGGAG (SEQ ID NO: 12) |
| hU1_R | CAGGGGGAAAGCGCGAACGCA (SEQ ID NO: 13) |
| LUNAR1_F | GGAGGCTGAGGCCGCCTGTT (SEQ ID NO: 14) |
| LUNAR1_R | AGGCTGCAGGGGAACAGGTCTT (SEQ ID NO: 15) |
| HOTTIP_F1 | CCTAAAGCCACGCTTCTTTG (SEQ ID NO: 16) |
| HOTTIP_R1 | TGCAGGCTGGAGATCCTACT (SEQ ID NO: 17) |
| linc94 Forward | CACGGCTGGGGAAGTACAAT (SEQ ID NO: 18) |
| linc94 reverse | TCGGGAATGAGTGTTCGTGG (SEQ ID NO: 19) |
| qPCR genomic DNA | |
| primers | |
| hIGF1Renh_F | GTGGTTTAGGGTGGGTGAGG (SEQ ID NO: 20) |
| hIGF1R_enhR | AAAAGCCCAGTCACCTGGAG (SEQ ID NO: 21) |
| LUNAR1pro_F | CGGGTGCACCTTCTGAATCT (SEQ ID NO: 22) |
| LUNAR1pro_R | TCCCCACAAGGAGAAGGGAA (SEQ ID NO: 23) |
| ACTBpro_F | AAAGGCAACTTTCGGAACGG (SEQ ID NO: 24) |
| ACTBpro_R | TTCCTCAATCTCGCTCTCGC (SEQ ID NO: 25) |
| 1inCXCR4_F | TGATGGTCTTGCCAGTCAGG (SEQ ID NO: 26) |
| inCXCR4_R | TTCGGAGCCTCGTCATATCC (SEQ ID NO: 27) |
| 3C primers | |
| LUNAR1_promoterBait_ | /56-FAM/AGGAGGAAA/ZEN/ATTGAGGGTGACTCAG |
| probe | (SEQ ID NO: 28) |
| LUNAR1_promoterBait_ | CAGGGCTAGTAGACTAGAAAGATA (SEQ ID NO: 29) |
| primer | |
| IGF1R enhancerBait_probe | /56-FAM/AGCCCATGA/ZEN/AAGCAGGTGTGAGTTC |
| | (SEQ ID NO: 30) |
| IGF1R enhancerBait_primer | CTCACCCACCCTAAACCACAGAAGCA (SEQ ID NO: 31) |
| enhancerUs5_R | TCCATGTGGGAGAGACAGTG (SEQ ID NO: 32) |
| enhancerUs4_R | TTTCAGTGAAGTCCCCCATC (SEQ ID NO: 33) |
| enhancerUs3_R | CCCACCTAACAATGATCCTGA (SEQ ID NO: 34) |
| enhancerDs1_R | CCTTCTCACCCATCGATCTC (SEQ ID NO: 35) |
| enhancerDs2_R | ATAGCCGGGCTTAGCTTCTC (SEQ ID NO: 36) |
| enhancerDs3_R | GCCCACCTCAGGCTTTACTT (SEQ ID NO: 37) |
| enhancerDs4_R | GATCAGCATTGTGAGCAGCA (SEQ ID NO: 38) |
| enhancerDs5_R | GGAGTCCAGCACAGTGTCAA (SEQ ID NO: 39) |
| controlPrimer | AGGGCTCCAATGCCAAATAAGTGTGG (SEQ ID NO: 40) |
| shRNA and ASO sequences | |
| shLUNAR1_1 | TGCTGTTGACAGTGAGCGcCAGTAGAGTGGTGGAAAG |
| | AGATAGTGAAGCCACAGATGTATCTCTTTCCACCACTC |
| | TACTGtTGCCTACTGCCTCGGA (SEQ ID NO: 41) |
| shLUNAR1_2 | TGCTGTTGACAGTGAGCGcCCGCCTGTTGAGTCACAGT |
| | TTTAGTGAAGCCACAGATGTAAAACTGTGACTCAACA |
| | GGCGGtTGCCTACTGCCTCGGA (SEQ ID NO: 42) |
| shMYC | TGCTGTTGACAGTGAGCGCCAGAATTTCAATCCTAGTA |
| | TATAGTGAAGCCACAGATGTATATACTAGGATTGAAAT |
| | TCTGTTGCCTACTGCCTCGGA (SEQ ID NO: 43) |
| shRenilla | TGCTGTTGACAGTGAGCGCAGGAATTATAATGCTTATC |
| | TATAGTGAAGCCACAGATGTATAGATAAGCAATAATT |
| | CCTATGTGCCTACTGCCTCGGA (SEQ ID NO: 44) |
| Shlinc94 | TGCTGTTGACAGTGAGCGcCACGGACTCCAGGACAGTT |
| | AATAGTGAAGCCACAGATGTATTAACTGTCCTGGAGTC |
| | CGTGtTGCCTACTGCCTCGGA (SEQ ID NO: 45) |
| ASO-Scr | mA*mA*mG*mC*mG*C*G*C*A*C*C*A*G*C*G*mC*mC* |
| | mU*mC*mC (SEQ ID NO: 46) |
| ASO1-LUNAR1 nucleotide | GGUCUUCTCCTCCAAACTGCUAAGC (SEQ ID NO: 71) |
| sequence | |
| ASO1-LUNAR1 w/ modified | mG*mG*mU*mC*mU*mU*C*T*C*C*T*C*C*A*A*A*C*T* |
| backbone | G*mC*mU*mA*mA*mG*mC (SEQ ID NO: 47) |
| ASO2-LUNAR1 nucleotide | CUCCCUTAGCCTTTCTAGTUUCCGU (SEQ ID NO: 72) |
| sequence | |
| ASO2-LUNAR1 w/ modified | mC*mU*mC*mC*mC*mU*T*A*G*C*C*T*T*T*C*T*A*G* |
| backbone | T*mU*mU*mC*mC*mG*mU (SEQ ID NO: 48) |
| ASO1-Lin-CXCR4 | UUGUUGGGTGAGTATGTGGUGGUGU (SEQ ID NO: 73) |
| nucleotide sequence | |
| ASO1-Lin-CXCR4 w/ | mU*mU*mG*mU*mU*mG*G*G*T*G*A*G*T*A*T*G*T*G |
| modified backbone | *G*mU*mG*mG*mU*mG*mU (SEQ ID NO: 49) |
| ASO2-Lin-CXCR4 | GGCUUUGTTGGGTGAGTATGUGGUGG (SEQ ID NO: 74) |
| nucleotide sequence | |
| ASO2-Lin-CXCR4 w/ | mG*mG*mC*mU*mU*mU*G*T*T*G*G*G*T*G*A*G*T*A |
| modified backbone | *T*G*mU*mG*mG*mU*mG*mG (SEQ ID NO: 50) |
| ChIRP probes | |
| LUNAR1_01 | aaactgtgactcaacaggcg/3BioTEG/ (SEQ ID NO: 51) |
| LUNAR1_02 | gccatctggttaaaatgagc/3BioTEG/ (SEQ ID NO: 52) |
| LUNAR1_03 | cctccaaactgctaagcaag/3BioTEG/ (SEQ ID NO: 53) |
| LUNAR1_04 | gaaggctgcaggggaacagg/3BioTEG/ (SEQ ID NO: 54) |
| LUNAR1_05 | catgagttgaggictaatca/3BioTEG/ (SEQ ID NO: 55) |
| LUNAR1_06 | tgtgagcaaggcaagagttc/3BioTEG/ (SEQ ID NO: 56) |
| LUNAR1_07 | ctatgagcatttactcccac/3BioTEG/ (SEQ ID NO: 57) |
| LUNAR1_08 | gttcctgggagatttctaga/3BioTEG/ (SEQ ID NO: 58) |
| LUNAR1_09 | tttctagtttccgtgatgtc/3BioTEG/ (SEQ ID NO: 59) |
| LUNAR1_10 | tagaggaagattggactccc/3BioTEG/ (SEQ ID NO: 60) |
| LacZ_01 | ccagtgaatccgtaatcatg/3BioTEG/ (SEQ ID NO: 61) |
| LacZ_02 | gtagccagctttcatcaaca/3BioTEG/ (SEQ ID NO: 62) |
| LacZ_03 | atcttccagataactgccgt/3BioTEG/ (SEQ ID NO: 63) |
| LacZ_04 | ataatttcaccgccgaaagg/3BioTEG/ (SEQ ID NO: 64) |
| LacZ_05 | ttcatcagcaggatatcctg/3BioTEG/ (SEQ ID NO: 65) |
| LacZ_06 | tgatcacactcgggtgatta/3BioTEG/ (SEQ ID NO: 66) |
| LacZ_07 | aaacggggatactgacgaaa/3BioTEG/ (SEQ ID NO: 67) |
| LacZ_08 | gttatcgctatgacggaaca/3BioTEG/ (SEQ ID NO: 68) |
| LacZ_09 | tgtgaaagaaagcctgactg/3BioTEG/ (SEQ ID NO: 69) |
| LacZ_10 | gtaatcgccatttgaccact/3BioTEG/ (SEQ ID NO: 70) |

mN = 2'-O-Me RNA base
*= Phosphorothioate Bonds

RNA-Sequencing Data Analysis. All RNA-seq data were aligned to hg19 using TopHat (Trapnell et al., “TopHat: Discovering Splice Junctions with RNA-Seq,” Bioinformatics 25:1105-1111 (2009), which is hereby incorporated by reference in its entirety) v1.4 with default parameters. Cuffdiff (Trapnell et al., “Transcript Assembly and Quantification by RNA-Seq Reveals Unannotated Transcripts and Isoform Switching During Cell Differentiation,” Nat. Biotechnol. 28:511-515 (2010), which is hereby incorporated by reference in its entirety) v1.3 was used for all differential expression (DE) analyses with a custom annotation consisting of RefSeq entries plus T-ALL lncRNAs as described below. In all DE tests, a gene was considered significant if the q value was less than 0.05 (Cuffdiff default).

lncRNA Discovery. Two samples from T-ALL cell lines (CUTLL1 and HPBALL) were sequenced, two primary human thymus samples to ultra-high depth (>200 million mate pairs each), ten primary pediatric T-ALL samples (60-80 million mate pairs each), and data generated by the Roadmap Epigenomics project for Naive CD4+ and CD8+ T cells to be used for ab initio transcriptome assembly with Cufflinks v1.3. Briefly, Cufflinks was run with the following options: -u, -N, -g (RefSeq GTF file provided as guide), and -M (rRNA and 7SK RNA mask file provided). Transcriptome assemblies were generated for each of these samples separately and then used Cuffmerge to combine all annotations. Any transcript that overlapped a known coding region (RefSeq NM entries) or T cell receptor and B cell receptor gene loci was removed in order to remove products of antigen receptor recombination. Next any transcript that did not overlap a region of enrichment for either H3K27ac, H3K4me1, or H3K4me3 in T-ALL or T cells, respectively, was removed and then merged the T-ALL and T cell annotations using Cuffmerge. Any gene locus that did not have at least one multi-exonic isoform or at least one isoform with >3× nucleotide coverage in one of our samples was removed. Any isoform with a length <200 nt was removed. Next, the PhyloCSF algorithm was used to predict protein-coding potential of the remaining transcripts. Multiple alignments were extracted for the 29 mammals supported by PhyloCSF using the Galaxy tools Extract multiple alignments and Stitch gene blocks. PhyloCSF was then run using -orf ATGStop to identify putative ORFs with a minimum ORF length of 50 amino acids. Any transcript with a PhyloCSF score greater than 100 was then removed as was previously used by Alvarez-Dominguez and colleagues (Alvarez-Dominguez et al., “Global Discovery of Erythroid Long Noncoding RNAs Reveals Novel Regulators of Red Cell Maturation,” Blood 123:570-581 (2014), which is hereby incorporated by reference in its entirety). Finally, all miRNA and snRNA host genes were removed. The resulting T-ALL lncRNA annotation was then compared to the GENCODE v18 lncRNA annotation in order to determine the number of novel genes discovered here. Transcripts were considered divergent if their TSS was less than 2.5 kb from a RefSeq NM TSS on the opposite strand. All other lncRNAs were considered to be intergenic. This T-ALL lncRNA annotation was merged with the RefSeqNM annotation using Cuffmerge and used for all subsequent RNA-Seq expression analyses.

ChIP. The following antibodies were used for chromatin immunoprecipitation experiments: Notch1 C-20 (Santa Cruz sc-6014), Med1 (Bethyl, A300-793A), Med12 (Bethyl, A300-774A), RNA PolII N-20 (Santa Cruz, sc-899), H3K4me1 (Abcam, ab8895), H3K4me3 (Active Motif, 39159), and H3K27ac (Abcam, ab4729). ChIP assays were performed essentially as described previously (Whyte et al., “Master Transcription Factors and Mediator Establish Super-enhancers at Key Cell Identity Genes,” Cell 153:307-319 (2013), which is hereby incorporated by reference in its entirety). Briefly, cells were crosslinked in 1% formaldehyde for 10 min at room temperature and the reaction was stopped by the addition of 0.125M glycine and incubated for an additional 5 min. Cells were washed twice with ice-cold PBS. Cells were then lysed in LB1 (50 mM HEPES, pH 7.5, 140 mM NaCl, 1 mM EDTA, 10% glycerol, 0.5% NP-40, 0.25% Triton-X) for 10 min at 4° C. and crude nuclei were pelleted by centrifugation. Nuclei were then resuspended in sonication buffer (20 mM Tris pH 8, 150 mM NaCl, 0.1% SDS, 1% Triton-X, 2 mM EDTA) and chromatin was sheared using a QSonica Q500 with microtip (40% amplitude, 15 s on 60 s off, total time 7.5 min). Following sonication, chromatin was cleared by high-speed centrifugation and supernatant was kept for immunoprecipitations. Antibodies (2.5 mg/IP for histones and 5 mg/IP for others) coupled to Protein-G Dynabeads (Life Technologies) were added to chromatin and incubated at 4° C. overnight. The following day, immune complexes were washed once with sonication buffer, once with high-salt wash buffer (20 mM Tris pH 8, 500 mM NaCl, 0.1% SDS, 1% Triton-X, 2 mM EDTA), once with lithium chloride wash buffer (10mMTris pH 8, 250mMLiC1, 1% NP-40), and once with TE supplemented with 50mMNaCl. Immunoprecipitates were then eluted by the addition of 1% SDS and 25mMsodium bicarbonate and incubated with shaking at 65° C. for 1 hr. Crosslinks were reversed by incubation at 65° C. for 6-18 hr and then treated with RNase A followed by Proteinase K. DNA was purified using QIAGEN PCR purification columns and used as template for qPCR.

ChIP-Seq Data Analysis. ChIP-seq for Notch1, Rbpjk, H3K4me3, H3K27ac, and p300 in CUTLL1 cells were performed previously (Wang et al., “NOTCH1-RBPJ Complexes Drive Target Gene Expression Through Dynamic Interactions with Superenhancers,” Proc. Natl. Acad. Sci. USA 111:705-710 (2014); Wang et al., “Genome-wide Analysis Reveals Conserved and Divergent Features of Notch1/RBPJ Binding in Human and Murine T-lymphoblastic Leukemia Cells,” Proc. Natl. Acad. Sci. USA 108:14908-14913 (2011a), which are hereby incorporated by reference in their entirety). RNAP2 ChIP-seq was preformed previously (King et al., “The Ubiquitin Ligase FBXW7 Modulates Leukemia-initiating Cell Activity by Regulating MYC Stability,” Cell 153:1552-1566 (2013), which is hereby incorporated by reference in its entirety). For all data sets, FASTQ files were aligned to hg19 using Bowtie with the following options: -n 2, -m 1. Resulting SAM files were then converted to BAM format and sorted by chromosomal location using Picard. Enriched regions were determined using MACS (Zhang et al., “Model-based Analysis of ChIP-Seq (MACS),” Genome Biol. 9:R137 (2008), which is hereby incorporated by reference in its entirety) v1.4 with a p value cutoff of 1e-9. ChIP-seq density heatmaps and histograms were generated using Genomic-Tools (Tsirigos et al., “GenomicTools: A Computational Platform for Developing High-throughput Analytics in Genomics,” Bioinformatics 28:282-283 (2012), which is hereby incorporated by reference in its entirety). lncRNAs were considered bound by Notch1 if they had an enriched region either within the gene or 5 kb up or downstream.

ChIRP. ChIRP assays were performed as described (Chu et al., “Genomic Maps of Long Noncoding RNA Occupancy Reveal Principles of RNA-chromatin Interactions,” Mol. Cell 44:667-678 (2011), which is hereby incorporated by reference in its entirety) with the following modifications. Cells were double crosslinked first with 2 mM EGS for 45 min at room temperature and washed twice with ice-cold PBS. Cells were then further cross-liked with 3% formaldehyde for 30 min at room temperature, and reaction was stopped by the addition of 0.125M glycine followed by two more washes in PBS. Crosslinked cells were then lysed in sonication buffer (see ChIP method) supplemented with SUPERaseIn (Life technologies), and chromatin was sheared exactly as for ChIP assays. Chromatin was cleared by centrifugation and supernatant was used for ChIRP reactions. All following steps were performed exactly as described by Chu et al. with the following exceptions: in the hybridization buffer, 1% Triton with 0.1% SDS was used instead of 1% SDS, and in the wash buffer, SDS concentration was lowered from 0.5% to 0.1%. Probes used for ChIRP assays are listed in Table 2.

Chromosome Conformation Capture. 3C experiments were performed essentially as described previously (Hagege et al., “Quantitative Analysis of Chromosome Conformation Capture Assays (3C-qPCR),” Nat. Protoc. 2:1722-1733 (2007), which is hereby incorporated by reference in its entirety). Briefly, 107 CUTLL1 cells per experiment were crosslinked with 1% formaldehyde in PBS for 10 min at room temperature and quenched by adding 0.125M glycine for 5 min. Cells were then washed twice in ice-cold PBS. Cells were then lysed in 3C lysis buffer (10 mM Tris-HCl, pH 7.5; 10 mM NaCl; 5 mM MgCl2; 0.1mMEGTA; 1× Sigma protease inhibitor cocktail) for 10 min on ice and nuclei were pelleted by centrifugation at 6003 g for 5 min. Pellets were then resuspended in 1× NEB buffer 2 with 1× BSA, 0.3% SDS and incubated with rotation at 37° C. for 1 hr. Triton X was then added and to a final concentration of 2% incubated again for 1 hr at 37° C. with rotation. 400U of highly concentrated HindIII (NEB, Ipswich, Mass., USA), was added and incubated overnight at 37_C with rotation. The following day SDS was added to a final concentration of 1.6% and samples were incubated at 65° C. for 20 min. Samples were then brought up to a final volume of 7 ml in 1× NEB T4 ligase buffer with 1× BSA and 1% Triton X. Samples were rotated at 37° C. for 1 hr. Samples were then chilled on ice for 5 min and 4000U T4 DNA ligase was added (NEB) and samples were incubated at 16° C. for 6 hr followed by 30 min at room temperature. Next, 300 mg of proteinase K was added, and crosslinks were reversed at 65° C. overnight. The following day, an additional 300 mg of proteinase K was added and incubated at 50° C. for 1 hr. Finally, genomic DNA was purified by phenol chloroform extraction followed by ethanol precipitation. Ligation events were detected using specific primers and Zen double quenched probes with 50 6-FAM and 30 Zen/Iowa black quenchers (IDT DNA, Coralville, Iowa, USA). qPCRs were performed on a Roche Lightcycler 480i using 23 Roche Probes mastermix. Specificity and efficiency of all 3C primes was verified by performing digestion and ligation of BACs containing the regions of interest. Ligation products were then serially diluted in sheared genomic DNA and the efficiency of each PCR reaction was verified. Amplicons from BAC qPCRs and actual 3C template were run on agarose gel to verify the production of a single band of the expected size.

RT-qPCR Analysis. For all RT-qPCR analyses, whole RNA was isolated using the QIAGEN RNeasy mini plus kit (Life Technologies, Carlsbad, Calif., USA). cDNA was generated using the High Capacity cDNA Reverse Transcription kit (Life Technologies). All qPCR primers were verified to produce specific primers and to perform efficiently. qPCR reactions were performed with technical triplicates on a Roche Lightcycler 480i with Roche 2× SYBR mastermix. Relative quantification of target genes was performed using the DDCt method with GAPDH as a reference gene.

RACE. 5′ and 3′ RACE were performed using the FirstChoice-RLM RACE kit according to the manufacturer's instructions (Life Technologies).

shRNA Design and Cloning. shRNAs were generated using the DSIR algorithm (http://biodev.extra.cea.fr/DSIR/DSIR.html). Briefly, FASTA sequences for each gene were provided to DSIR using the 21 nt siRNA setting. The top 10 ranking siRNAs were taken and the reverse complement from the “AS sequence” output field was used as the basis for designing 97-mer template oligos at: http://katandin.cshl.org/siRNA/RNAi.cgi?type=shRNA. 97 mers were synthesized, diluted to 0.1 ng/ml, and mixed into a pool. Pools were then used as template for a PCR reaction which added XhoI and EcoRI sites for cloning into LMP vectors. PCR products were purified using QIAGEN PCR cleanup kit and digested with EcoRI and XhoI and subsequently ligated into EcoRI/XhoI-digested LMP vector. Ligation products were transformed into E. coli and clones were screened for correct inserts.

Luciferase Reporter Assays. 293T or 293-UAS-TK-Luc cells were grown until 60% confluent and transfected with indicated plasmids plus pTK-Ren using polyethylenimine (PEI). For all experiments as the amount of BoxB-lncRNA plasmid was decreased, an equal amount of BoxB-Empty vector was added in its place. Twenty-four hours after transfection, cells were harvested and assayed for reporter activity using the Dual-Glo Luciferase Assay System and a GloMax-Multi Jr. luminometer according to manufacturer's instructions (Promega, Madison, Wis., USA). Each data point was taken as the average Luc/Ren ratio of 3 triplicate wells.

Xenograft In Vivo Competition Assay. CUTLL1 cells were transduced with retroviruses expressing either LMP-shRenilla-GFP or LMP-shLUNAR-mCherry. Forty-eight hours following the infection, puromycin was added to culture media until cultures were >95% GFP or mCherry positive and >90% alive. GFP and mCherry cells were then mixed 1:1 and the relative contribution of cells containing each shRNA was verified by FACS. This mixture was then xenografted into sublethally irradiated (350 cGy) Rag2^{-/-}IL2Rg^{-/-} animals at 1 million cells per animal via tail vein injection. After 4 weeks, animals were sacrificed and spleens were harvested and stained with anti-human CD45 to mark human leukocytes. We then measured the relative contribution of cells harboring each shRNA by FACS analysis.

Hi-C Data Analysis. Hi-C sequencing data were preprocessed (iterative alignment and outlier removal) using the pipeline described by Imakaev and colleagues (Imakaev et al., “Iterative Correction of Hi-C Data Reveals Hallmarks of Chromosome Organization,” Nat. Methods 9:999-1003 (2011), which is hereby incorporated by reference in its entirety). The heatmap in the IGF1R/LUNAR1 locus shows the Hi-C interactions as paired-read counts between pairs of sliding windows of 50 kb in length.

ASO Design and Delivery. Antisense oligonucleotides (ASOs) were designed using the IDT Antisense Design Tool (http://www.idtdna.com) using the chimeric 25-mer setting. The top 3 ASOs generated by the design tool were ordered and tested for knockdown efficiency. The ASO with the highest potency was used for experiments. Sequence for the scrambled ASO was taken from Li et al., “Functional Roles of Enhancer RNAs for Oestrogen-dependent Transcriptional Activation,” Nature 498:516-520 (2013), which is hereby incorporated by reference in its entirety. For ASO experiments in T-ALL cells oligonucleotides were delivered simply by adding them to the growth media at 2 mM. For ASO knockdown in BoxB tethering experiments, ASOs were cotransfected with plasmid DNA at 50 nM.

Accession Numbers. The NCBI Gene Expression Omnibus accession number for the RNA-Seq data reported in this paper is GSE57982.

### Example 1

### Comprehensive Mapping of lncRNAs in T-ALL

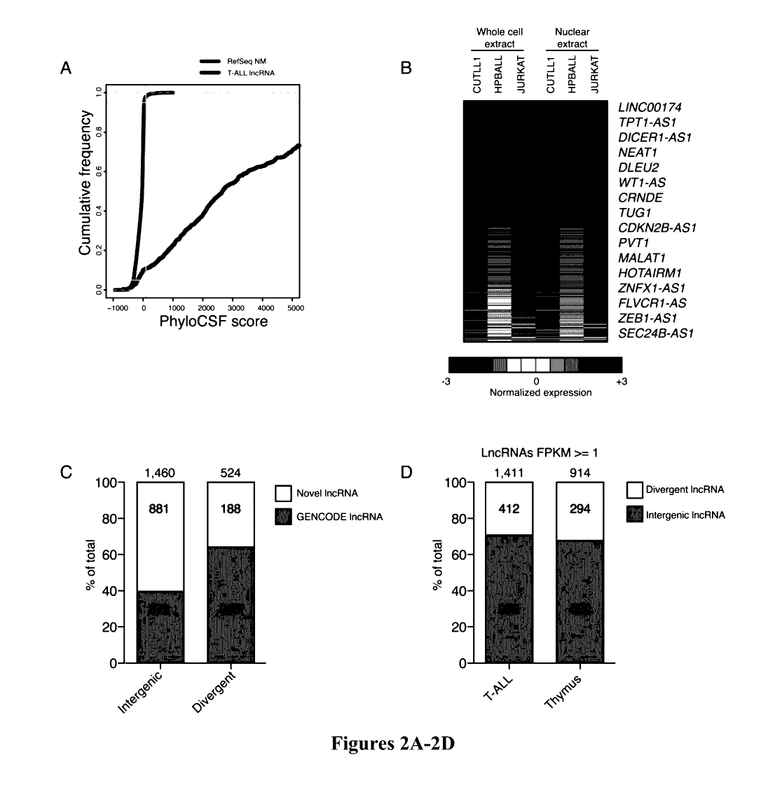

To gain insights into possible functional roles for lncRNAs in T-ALL, it was first sought to create a high-quality map of such transcripts for use in subsequent analyses and functional studies. This goal was achieved by utilizing the workflow outlined in FIG. 1A. Briefly, ultra-high-depth RNA-sequencing (RNA-seq) data sets were generated from multiple human T-ALL cell lines and primary leukemia samples. These data were then used to generate the most comprehensive T-ALL transcriptome assembly to date using an “align then assemble” (Trapnell et al., “TopHat: Discovering Splice Junctions with RNA-Seq,” Bioinformatics 25:1105-1111(2009), which is hereby incorporated by reference in its entirety) approach. To isolate only putative lncRNA genes, all known protein-coding genes were removed and retained all transcripts previously reported as lncRNAs or that have not been identified thus far in other annotation efforts. Single-exon transcripts were eliminated to focus on products of splicing events. Finally, because lncRNA transcription has been suggested to occur by a mechanism very similar to that of coding genes (Guttman et al., “Chromatin Signature Reveals Over a Thousand Highly Conserved Large Non-coding RNAs in Mammals,” Nature 458:223-227 (2009), which is hereby incorporated by reference in its entirety), any transcript that did not display enrichment of promoter-associated histone modifications (H3K4me3, H3K4me1, H3K27ac) was removed. These efforts have yielded a high confidence T-ALL lncRNA annotation comprised of 6,023 isoforms derived from 1,984 unique gene loci. Out of all of these loci, 46% were identified in the GENCODE v18 lncRNA annotation (Harrow et al., GENCODE: The Reference Human Genome Annotation for The ENCODE Project,” Genome Res. 22:1760-1774 (2012), which is hereby incorporated by reference in its entirety) (FIG. 1B), suggesting the presence of many novel lncRNAs in T-ALL. Additionally, it was observed that approximately 26% of lncRNAs detected here were expressed in a divergent orientation with respect to protein-coding genes, whereas the remaining 74% were true intergenic gene loci with their own regulatory elements (FIG. 1C), and this ratio of divergent to intergenic lncRNAs is approximately the same in T-ALL and normal T cell progenitors (FIG. 2D). Additionally, 40% of intergenic lncRNAs were identified by GENCODE, whereas the remaining 60% represent novel lncRNA loci (FIG. 2C available online).

The expression patterns of the T-ALL lncRNA catalog were next examined over a diverse panel of cell types including T-ALL, primary T cells, and many other somatic tissues (Human Body Map data). In agreement with previous reports (Cabili et al., “Integrative Annotation of Human Large Intergenic Noncoding RNAs Reveals Global Properties and Specific Subclasses,” Genes Dev. 25:1915-1927 (2011), which is hereby incorporated by reference in its entirety), it was observed that average lncRNA expression was lower than protein-coding gene expression (FIG. 1D). Additionally, putative lncRNAs showed very little protein-coding potential as measured by the PhyloCSF algorithm (Lin et al., “PhyloCSF: A Comparative Genomics Method to Distinguish Protein Coding and Non-coding Regions,” Bioinformatics 27:i275-i282 (2011), which is hereby incorporated by reference in its entirety) (FIG. 2A). In agreement with possible roles in regulating gene expression, many lncRNAs were enriched in RNA from nuclear extracts compared to total RNA (FIG. 2B). Finally, upon examination of the 250 most variable lncRNAs, as measured by quartile coefficient of dispersion, striking specificity for T-ALL and normal T cells (FIG. 1E) was observed, which is consistent with the notion (Guttman et al., “Chromatin Signature Reveals Over a Thousand Highly Conserved Large Non-coding RNAs in Mammals,” Nature 458:223-227 (2009), which is hereby incorporated by reference in its entirety) that lncRNA expression is highly tissue specific. However, many lncRNAs were specifically expressed in human T-ALL when compared to untransformed peripheral T cells (FIG. 1E), suggesting that T cell transformation is dynamically changing the lncRNA landscape in this cell type. Overall, this effort cataloged T-ALL-specific noncoding RNAs and represents a comprehensive mapping of lncRNA expression in this aggressive subtype of ALL.

### Example 2

### T-ALL-Specific lncRNAs are Part of the NOTCH1 Oncogenic Network

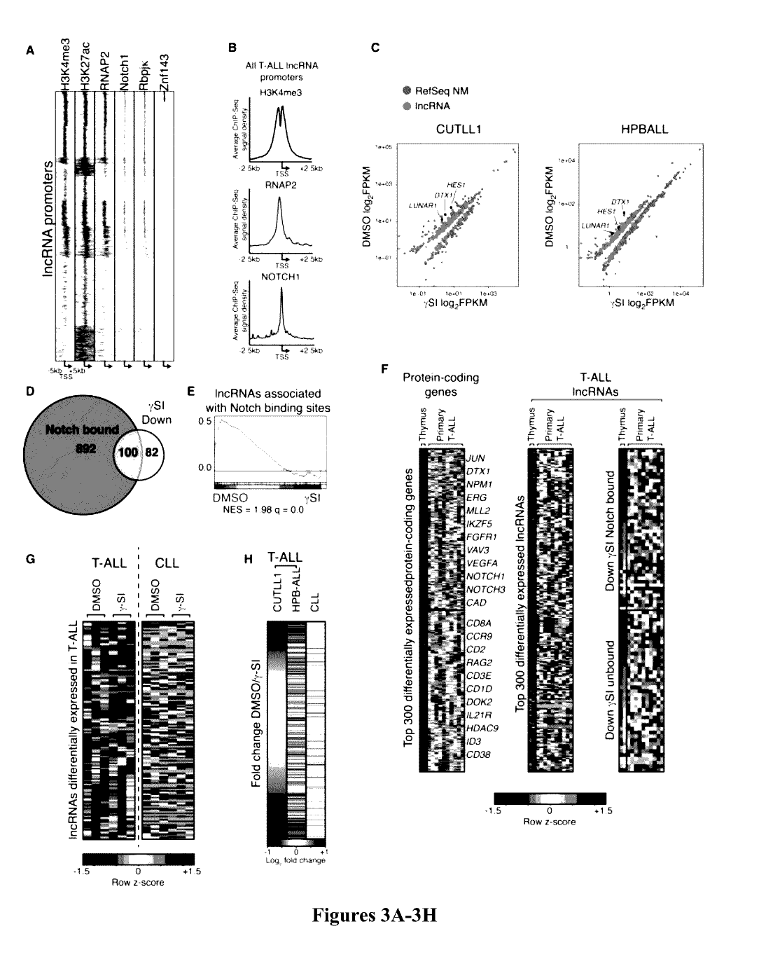

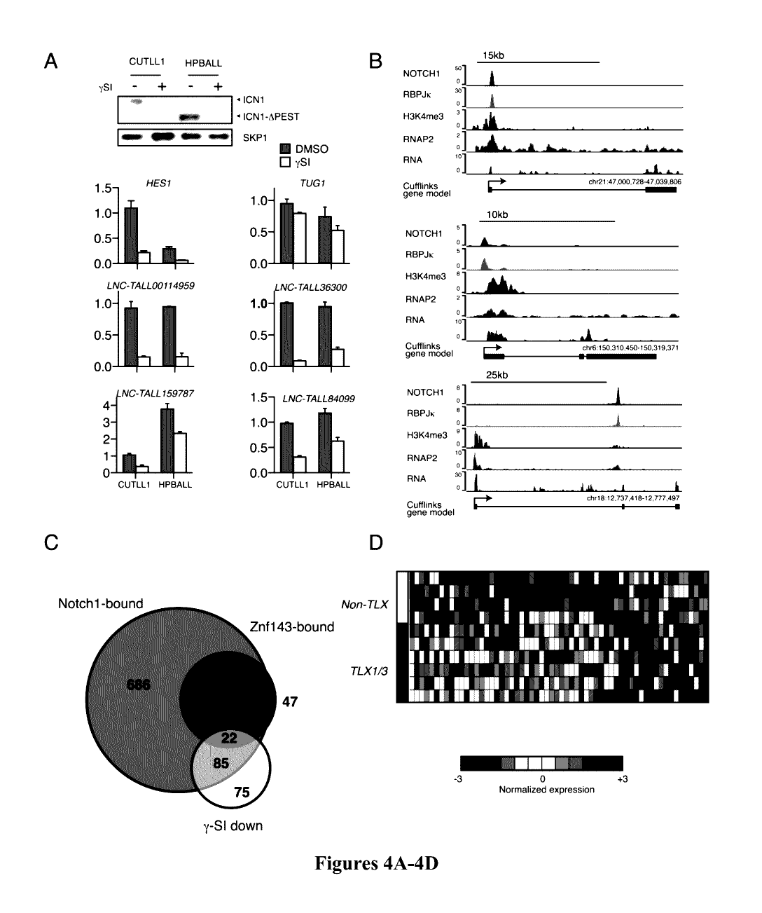

Given that oncogenic NOTCH1 activity is one of the key features in T-ALL and NOTCH pathway activity characterizes the vast majority (>90%) of human T-ALL cases, it was reasoned that the lncRNA expression program in this disease may be influenced by the NOTCH1/RBPJκ transcriptional activator complex. Upon examination of lncRNA promoters, a high density of NOTCH1/RBPJκ chromatin immunoprecipitation sequencing (ChIP-seq) signal was noticed at many of these regions (FIGS. 3A and 3B). Additionally, most lncRNA promoters displayed high enrichment of H3K4me3, H3K27ac, and RNA polymerase II (RNA PolII) signal proximal to their TSSs. A subset of lncRNAs was also observed, which showed co-occupancy of NOTCH1 and ZNF143 (FIGS. 3A and 4C), a protein that has been previously reported to co-occupy the genome with Notch, although its role in gene regulation is unclear (Wang et al., “Genome-wide Analysis Reveals Conserved and Divergent Features of Notch1/RBPJ Binding in Human and Murine T-lymphoblastic Leukemia Cells,” Proc. Natl. Acad. Sci. USA 108:14908-14913 (2011a), which is hereby incorporated by reference in its entirety). It was therefore hypothesized that expression of a subset of lncRNA genes may be dependent on NOTCH1-mediated signaling. To test this hypothesis, chemical inhibition of the g-secretase complex (Kopan et al., “The Canonical Notch Signaling Pathway: Unfolding the Activation Mechanism,” Cell 137:216-233 (2009), which is hereby incorporated by reference in its entirety) was used to perturb NOTCH1 cleavage and nuclear translocation in two prototypical human T-ALL cell lines (CUTLL1 and HPB-ALL) and RNAseq was carried out to measure lncRNA expression following treatment and NOTCH inhibition. Such approaches have been extensively used in T-ALL and are proven to specifically target NOTCH1 signaling (Palomero et al., “CUTLL1, a Novel Human T-cell Lymphoma Cell Line with t(7;9) Rearrangement, Aberrant NOTCH1 Activation and High Sensitivity to Gamma-secretase Inhibitors,” Leukemia 20:1279-1287(2006); Weng et al., “Activating Mutations of NOTCH1 in Human T Cell Acute Lymphoblastic Leukemia,” Science 306:269-271 (2004), which are hereby incorporated by reference in their entirety). Indeed, it was observed that in addition to protein-coding genes, many lncRNA genes were differentially expressed upon g-secretase inhibitor (g-SI) treatment compared to vehicle controls (FIGS. 3C and 4A), suggesting direct regulation and possible roles downstream of NOTCH1 activation in this disease. In agreement with this notion, it was observed that approximately 55% of lncRNAs whose expression was Notch dependent were also directly occupied by NOTCH1 (FIG. 3D). In support of the hypothesis that NOTCH1 controls the lncRNA transcriptional program in T-ALL, it was observed that lncRNAs associated with the top 1,000 most enriched NOTCH1-binding sites were significantly downregulated upon administration of g-SI according to gene set enrichment analysis (GSEA) (FIG. 3E). Upon closer examination of many of these lncRNA loci, strong NOTCH1/RBPJk-binding sites (Wang et al., “Genome-wide Analysis Reveals Conserved and Divergent Features of Notch1/RBPJ Binding in Human and Murine T-lymphoblastic Leukemia Cells,” Proc. Natl. Acad. Sci. USA 108:14908-14913 (2011a), which is hereby incorporated by reference in its entirety) were observed at both promoters and intragenic enhancer elements (FIG. 4B, highlighted yellow), suggesting direct transcriptional control by Notch signaling. Together these data suggest the presence of a Notch-dependent T-ALL lncRNA expression program, members of which may carry important biological functions.

To address whether these Notch-regulated lncRNA genes might be expressed in primary NOTCH1-induced T-ALL, their expression was measured across ten patient samples that harbored activating NOTCH1 mutations as well as two primary human thymus samples, as a matched physiological tissue with low Notch activation (Ntziachristos et al., “Genetic Inactivation of the Polycomb Repressive Complex 2 in T Cell Acute Lymphoblastic Leukemia,” Nat. Med. 18:298-301 (2012), which is hereby incorporated by reference in its entirety). These analyses yielded a subset of lncRNAs that displayed differential expression in primary T-ALL compared to normal thymic T cells (FIG. 3F). It was also observed that a subset of lncRNAs are differentially expressed among distinct subtypes of T-ALL as defined by aberrant expression of TLX1 or TLX3 (FIG. 4D), suggesting a possible role in specifying discreet subclasses within this disease.

Given that activating mutations on NOTCH1 have been recently discovered in chronic lymphocytic leukemia (CLL) (Fabbri et al., “Analysis of the Chronic Lymphocytic Leukemia Coding Genome: Role of NOTCH1 Mutational Activation,” J. Exp. Med. 208:1389-1401 (2011), which is hereby incorporated by reference in its entirety), a tumor of the B-lymphocyte compartment, it was hypothesized that Notch-dependent lncRNAs that have been described in T-ALL might also show similar expression patterns in CLL. To test this hypothesis, Notch mutant CLL cells were cultured on OP9-DL1 stromal cells, which express the Notch ligand delta-like 1 (DL1), and added vehicle control (DMSO) or g-SI followed by RNA-seq (FIG. 3G). By comparing the T-ALL and CLL g-SI treatment experiments, it was observed that Notch-dependent changes in lncRNA expression in T-ALL are different from those in CLL (FIGS. 3G and 3H). It was observed very little conservation of the Notch-dependent T-ALL lncRNA expression program in Notch mutant CLL cells, suggesting that although these two tumors harbor similar oncogenic lesions, the downstream consequences of Notch activation are distinct.

### Example 3

### LUNAR1 is a NOTCH-Regulated lncRNA Transcript in Human T-ALL

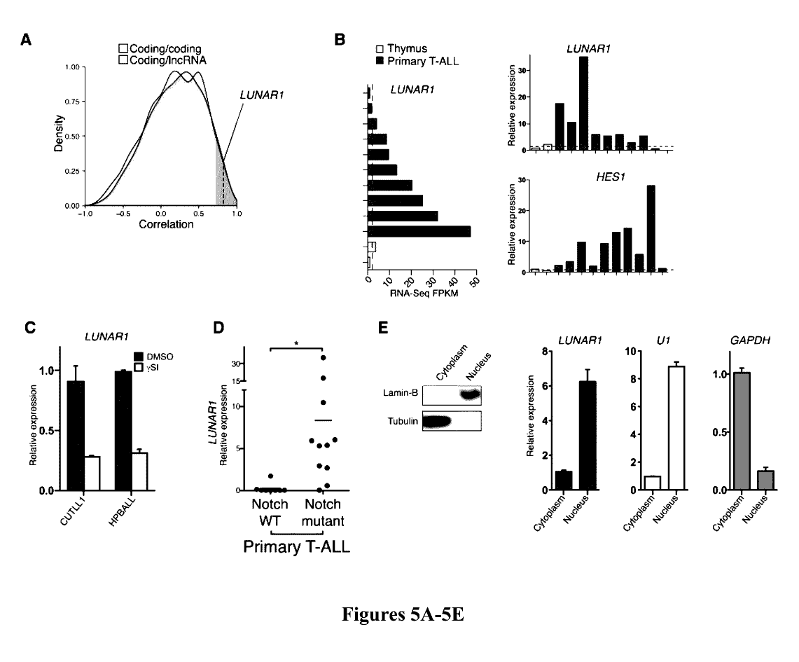

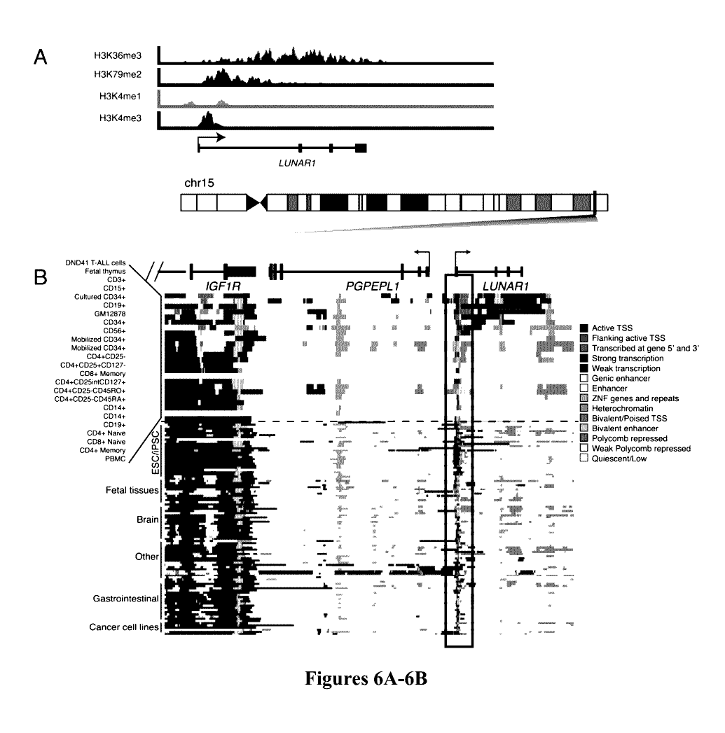

Because lncRNAs have been previously shown to enhance expression of nearby genes through cis-regulation (Gomez et al., “The NeST Long ncRNA Controls Microbial Susceptibility and Epigenetic Activation of the Interferon-g Locus,” Cell 152:743-754 (2013); Lai et al., “Activating RNAs Associate with Mediator to Enhance Chromatin Architecture and Transcription,” Nature 494:497-501 (2013); Ørom et al., “Long Noncoding RNAs with Enhancer-like Function in Human Cells,” Cell 143:46-58 (2010), which are hereby incorporated by reference in their entirety), it was next asked whether any of the lncRNAs identified here display a high Pearson correlation with neighboring protein-coding genes. By comparing correlation density plots for all coding/lncRNA and coding/coding gene pairs, it was found that in general, lncRNAs are no more correlated with neighboring genes than their coding counterparts (FIG. 5A). However, because enhancer-like activity for lncRNAs has been proposed before, all lncRNAs with at least 0.75 correlation were considered (FIG. 5A, shaded area) with a coding neighbor as candidates for further study as cis-regulators. From these candidates, one lncRNA gene was noticed, which was termed LUNAR1 (leukemia-induced noncoding activator RNA), that showed high correlation with its coding neighbor gene the insulin-like growth factor receptor 1 (IGF1R) (r=0.77), a gene which has been previously suggested to play a role in T-ALL (Medyouf et al., “High-level IGF1R Expression is Required for Leukemia-initiating Cell Activity in T-ALL and is Supported by Notch Signaling,” J. Exp. Med. 208:1809-1822 (2011), which is hereby incorporated by reference in its entirety). Several other features were noted that made LUNAR1 appear attractive as a candidate. It was downregulated upon Notch inhibition (FIG. 5C), overexpressed in primary T-ALL (FIG. 5B), and expressed significantly higher in T-ALL samples that harbored a Notch mutation compared to those without mutations (FIG. 5D). Additionally, an enrichment of LUNAR1 was also found in the nucleus (FIG. 5E), to a degree similar to that of U1, a component of a small nuclear ribonucleoprotein (snRNP) splicing complex, which supported the hypothesis that this transcript may be involved in gene regulation. By examining the chromatin state at the LUNAR1 locus, typical features of RNAP2-dependent genes in T-ALL were observed (FIG. 6A), as well as a transcriptionally active state in several related hematopoietic cell types, according to the chromHMM algorithm (FIG. 6B). Although a transcriptionally active chromatin state was restricted to very few samples, nearly all of the cell types for which chromHMM data were available showed signs of active promoter-associated chromatin structure proximal to the LUNAR1 TSS, indicating a true transcriptional unit (FIG. 6B). Although the LUNAR1 locus shows signs of promoter activity in diverse tissues, its expression is highly restricted, suggesting that specific factors are required for its activation. Using standard 50 and 30 rapid amplification of cDNA ends (RACE) approaches, a 491-nucleotide transcript containing 4 exons and a poly(A) tail (FASTA sequence in supplement) was cloned. In order to address whether LUNAR1 is indeed noncoding the PhyloCSF algorithm was utilized (Lin et al., “PhyloCSF: A Comparative Genomics Method to Distinguish Protein Coding and Non-coding Regions,” Bioinformatics 27:i275-i282 (2011), which is hereby incorporated by reference in its entirety), which yielded a score of −48.12, indicating lack of an open reading frame (ORF) with selective pressure for codon preservation. Additionally, PFAM was used to translate the RNA sequence in three frames and searched for known protein domains, of which none were found (not shown). These data strongly suggest that the LUNAR1 transcript is unlikely to encode any protein product.

### Example 4

### LUNAR1 Expression is Controlled by an Intronic Enhancer in the IGF1R Locus

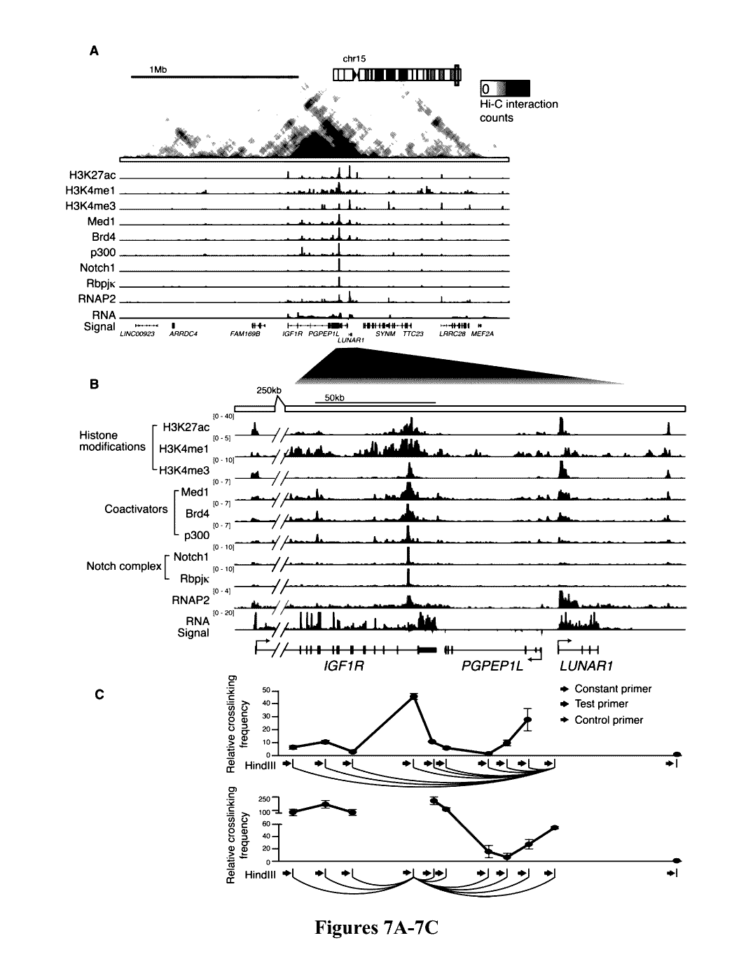

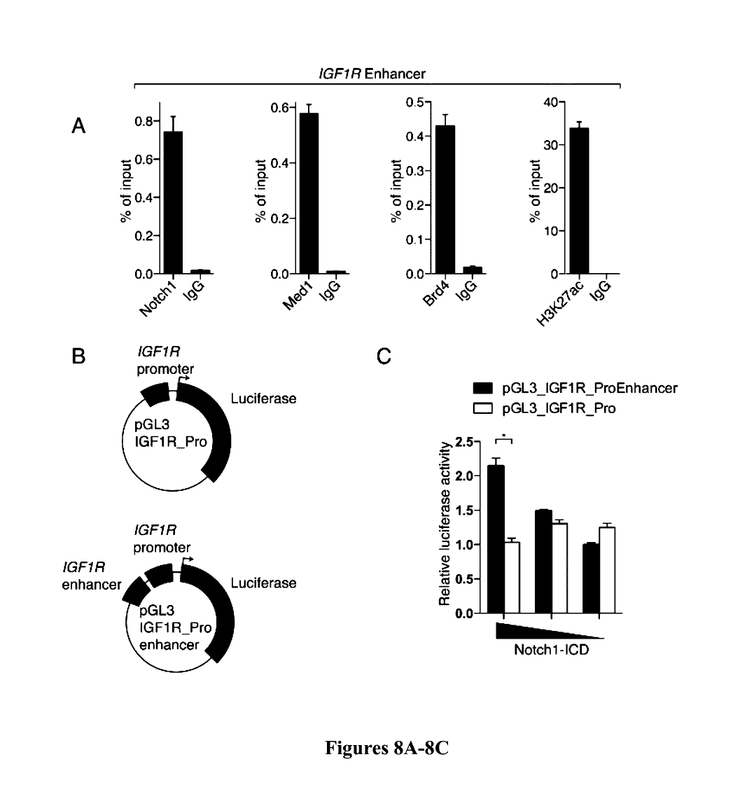

To further study possible functional significance of LUNAR1, genome-wide chromosome conformation capture (Hi-C) was utilized in T-ALL cells (CUTLL1) to examine the higher-order chromatin context in T-ALL. Similar to other chromosome capture methods, Hi-C utilizes restriction enzyme digestion of crosslinked chromatin followed by intramolecular ligation to physically link genomic regions that were in close spatial proximity to one another (Lieberman-Aiden et al., “Comprehensive Mapping of Long-range Interactions Reveals Folding Principles of the Human Genome,” Science 326:289-293 (2009), which is hereby incorporated by reference in its entirety). By coupling this method to high-throughput sequencing, a map of 3D chromatin structure in human T-ALL cells was created. In doing so, it was discovered that LUNAR1 resides in a 500 kb topologically associating domain (Dixon et al., “Topological Domains in Mammalian Genomes Identified by Analysis of Chromatin Interactions,” Nature 485:376-380 (2012), which is hereby incorporated by reference in its entirety) on chromosome 15, which also includes neighboring genes IGF1R and PGPEP1L (FIG. 7A). Of these two neighboring genes, only IGF is transcriptionally active; therefore it was reasoned that LUNAR1 might play a role in enhancing expression of this gene. Closer examination of the LUNAR1/IGF1R locus revealed the presence of a highly active enhancer in the last intron of IGF1R, which showed a high degree of occupancy by NOTCH1 (previously reported; Medyouf et al., “High-level IGF1R Expression is Required for Leukemia-initiating Cell Activity in T-ALL and is Supported by Notch Signaling,” J. Exp. Med. 208:1809-1822 (2011), which is hereby incorporated by reference in its entirety), Mediator subunit MED1, histone acetyltransferase P300, RNA PolII, and the histone reader BRD4 (FIGS. 7B and 8A), all of which have been suggested to be hallmarks of active enhancer elements when located outside of promoter regions (Heintzman et al., “Histone Modifications at Human Enhancers Reflect Global Cell-type-specific Gene Expression,” Nature 459:108-112 (2009); Rada-Iglesias et al., “A Unique Chromatin Signature Uncovers Early Developmental Enhancers in Humans,” Nature 470:279-283 (2011); Whyte et al., “Master Transcription Factors and Mediator Establish Super-enhancers at Key Cell Identity Genes,” Cell 153:307-319 (2013), which are hereby incorporated by reference in their entirety). In addition to showing enrichment for enhancer-associated chromatin factors and a chromatin signature characteristic of active enhancer elements (high H3K4me1, low H3K4me3, high H3K27ac), it was shown that this element is able to drive expression in a reporter assay in a Notch-dependent manner (FIGS. 8B and 8C).

Chromosome conformation capture (3C) was used followed by quantitative PCR (qPCR) to validate the Hi-C findings and identified a peak of high crosslinking frequency at the IGF1R enhancer when using a constant HindIII fragment located close to the LUNAR1 promoter, indicating the presence of a chromatin loop that places these regions in close physical proximity within the 3D organization of the nucleus (FIG. 7C). This finding was verified using a constant HindIII fragment in the IGF1R enhancer, suggesting a specific interaction (FIG. 7C, lower panel). These results suggest that this Notch-occupied enhancer element in the IGF1R locus is able to control LUNAR1 expression through promoter/enhancer contacts.

### Example 5

### LUNAR1 Controls IGF1R Expression and is Essential for T-ALL Maintenance

To provide evidence further supporting a more causal relationship between

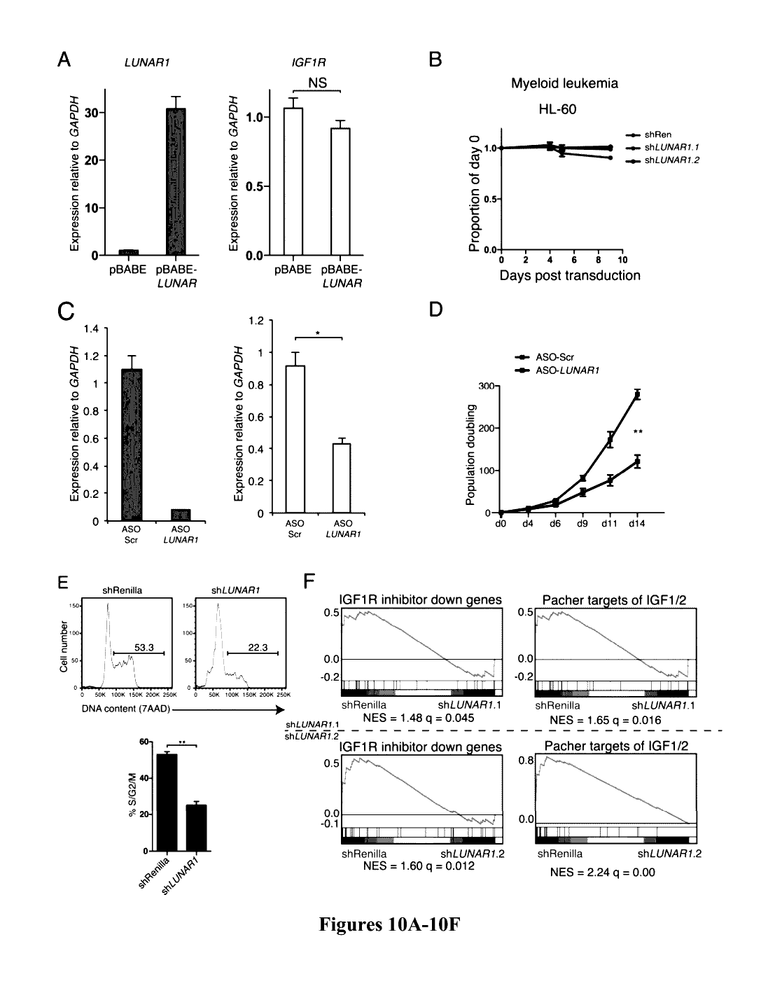

LUNAR1 and IGF1R, RNAi was used to attenuate LUNAR1 expression. In doing so, it was noticed that abrogation of LUNAR1 led to repression of IGF1R, an effect that was observed using two independent small hairpin RNAs (shRNAs) targeting distinct regions of the transcript (FIG. 9A). Ectopic expression of LUNAR1 using retroviral vectors, which integrate randomly into the genome, did not yield significantly different expression of IGF1R mRNA (FIG. 10A), supporting the hypothesis of a cis-activation mechanism. Additionally, LUNAR1 depletion resulted in a competitive growth disadvantage phenotype in T-ALL cells (CUTLL1, HPB-ALL) in which the transcript is highly expressed (FIG. 9B) but did not lead to a similar phenotype in myeloid leukemia cells (HL-60) (FIG. 10B), in which LUNAR1 expression is limited, suggesting on-target and T-ALL-specific effects (FIG. 9B). Depletion of LUNAR1 caused a significant decrease in the number of actively cycling cells as shown by 7AAD staining (FIG. 10E). These T-ALL growth effects were similar to the ones noted when the IGF1R gene was silenced (Medyouf et al., “High-level IGF1R Expression is Required for Leukemia-initiating Cell Activity in T-ALL and is Supported by Notch Signaling,” J. Exp. Med. 208:1809-1822 (2011), which is hereby incorporated by reference in its entirety). In order to further rule out off-target effects of RNAi, antisense DNA/RNA hybrid oligonucleotide (ASO) gene-silencing technology was used, which triggers RNase-H-mediated degradation of the target transcript upon ASO binding. ASO-mediated depletion of LUNAR1 led to both repression of IGF1R mRNA (FIG. 10C) and a growth retardation phenotype similar to what we observed with RNAi (FIG. 10D).

In order to test the effects of LUNAR1 depletion on tumor growth in vivo, xenograft assays were performed in which human T-ALL cells expressing an shRNA targeting LUNAR1 (GFP) or Renilla (mCherry) control were mixed at a 1:1 ratio and transferred intravenously into sublethally irradiated immunodeficient hosts (FIG. 9C). Four weeks after the transplantation, peripheral tumors were harvested and measured by fluorescence-activated cell sorting (FACS) analysis the relative contribution of cells harboring each shRNA. Using this assay, a significant loss of representation of cells in which LUNAR1 was depleted was observed (FIG. 9D), which suggested that this lncRNA is required for efficient tumor growth both in vitro and in vivo.

To test whether IGF1R is the relevant target of LUNAR1, ASO-mediated lncRNA depletion was performed in T-ALL cells ectopically expressing IGF using retroviral constructs or empty vector control. Following ASO delivery, it was observed that ectopic expression of IGF1R efficiently rescued the growth defects observed following LUNAR1 depletion (FIG. 9E), suggesting that IGF1R is indeed a key target of this lncRNA.

To characterize the overall cellular response to LUNAR1 silencing, global gene-expression analysis (RNA-seq) was performed in T-ALL cells following LUNAR1 depletion with two independent shRNAs. For comparison, experiments were also performed in which IGF1 signaling was blocked using pharmacological inhibition (BMS-536924) followed by RNA-seq. The outcome of these studies was striking as significant repression of IGF mRNA was noted, in agreement with qPCR studies (FIG. 9A). A subset of genes was also identified, which were similarly regulated following either IGF1R inhibition or LUNAR1 depletion (FIG. 9F), suggesting that this lncRNA promotes IGF1 signaling by transcriptional regulation of IGF1R. Finally, using GSEA, significant enrichment was found for gene sets containing genes significantly downregulated upon IGF1 inhibition in T-ALL (FIG. 10F) and other publicly available gene sets containing targets of IGF1/2 (FIG. 10F).

### Example 6

### LUNAR1 is an Activator RNA Capable of Stimulating Gene Activity

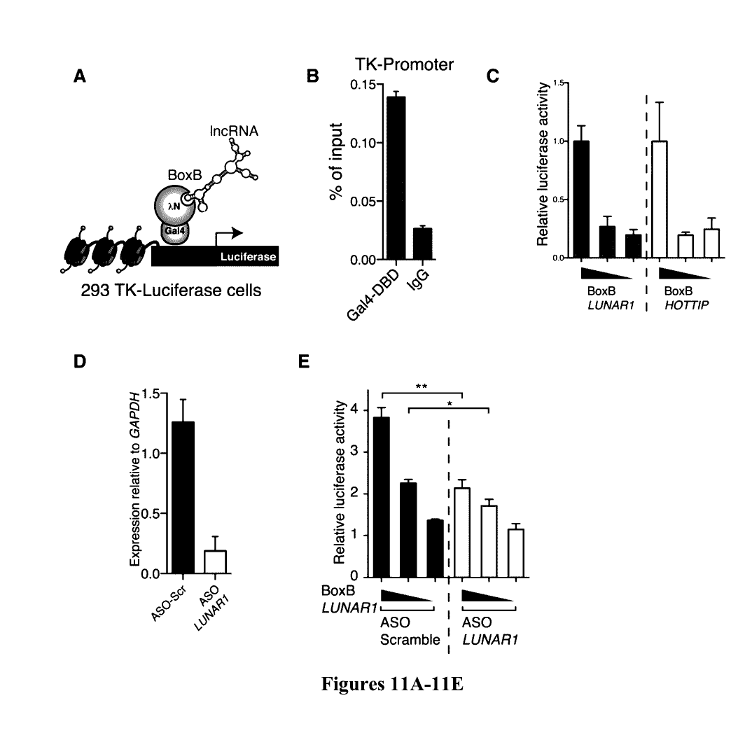

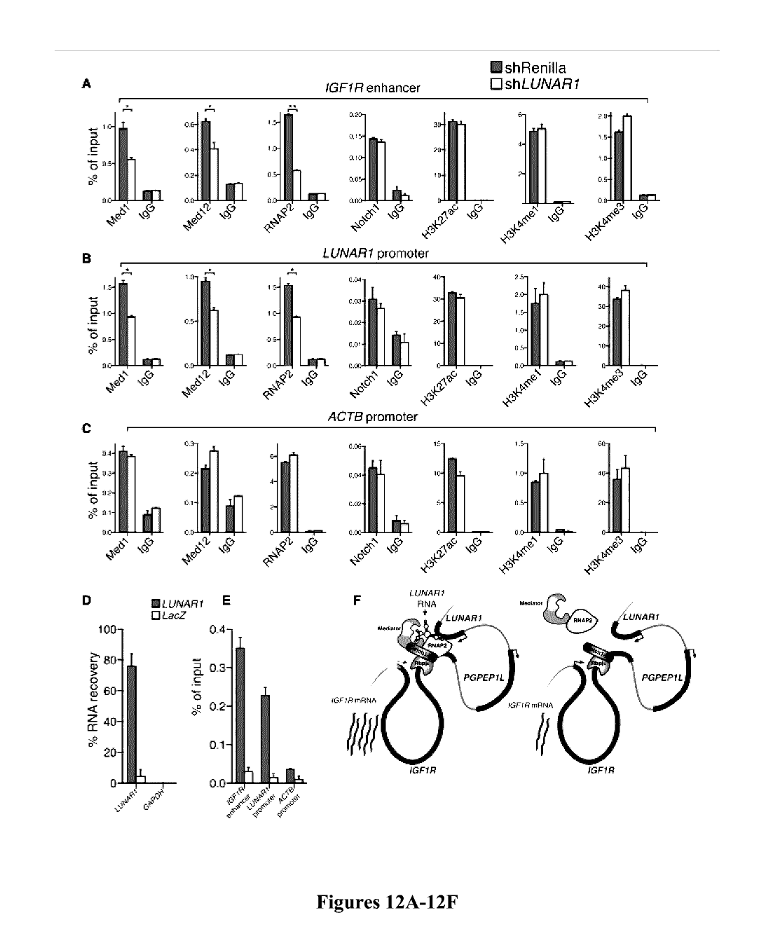

In order to test whether LUNAR1 displayed intrinsic ability to promote gene activity, the Gal4-1N/BoxB system was used to tether this lncRNA to a heterologous reporter promoter (Li et al., “Functional Roles of Enhancer RNAs for Oestrogen-dependent Transcriptional Activation,” Nature 498:516-520 (2013); Wang et al., “A Long Noncoding RNA Maintains Active Chromatin to Coordinate Homeotic Gene Expression,” Nature 472:120-124 (2011b), which are hereby incorporated by reference in their entirety). In this system, the BoxB RNA stem loop is fused to a lncRNA, which allows it to bind specifically and with high affinity to the phage 1 N-peptide (1N). By fusing 1N to the DNA-binding domain of the yeast Gal4 protein, a BoxB-tagged lncRNA can be tethered to UAS DNA sequences (FIG. 11A). Vector containing the Gal4-1N fusion was cotransfected with either BoxB-tagged LUNAR1 or known activator lncRNA HOTTIP into HEK293 cells stably expressing five UAS sites upstream of a TK-luciferase reporter gene (Vaquero et al., “Human SirT1 Interacts with Histone H1 and Promotes Formation of Facultative Heterochromatin,”Mol. Cell 16:93-105 (2004), which is hereby incorporated by reference in its entirety). Upon transfection, binding of the Gal4-1N fusion at the reporter promoter was detected as expected (FIG. 11B). Tethering LUNAR1 to this reporter gene stimulated transcription of the reporter to a similar degree as HOTTIP (FIG. 11C). Additionally, ASO-mediated depletion of LUNAR1 (FIG. 11D) significantly reduced this stimulatory effect compared to a non-targeting ASO (FIG. 11E), suggesting that molecules of LUNAR1 are functionally important for the stimulation of transcription seen here. In order to investigate the mechanism by which endogenous LUNAR1 promotes transcriptional activity of the IGF1R gene, chromatin studies were performed following depletion of the lncRNA in T-ALL cells. Because it was hypothesized that LUNAR1 might be an important component of the intronic IGF1R enhancer, it was reasoned that depletion of this transcript might influence chromatin state or occupancy of one of the activators at that locus. Following depletion of LUNAR1, a significant reduction in Mediator complex (MED1 and MED12) and RNA PolII occupancy was observed at both the IGF1R enhancer and the LUNAR1 promoter (FIGS. 12A and 12B). Additionally, significant loss of RNA PolII binding at the IGF1R promoter was observed (not shown). No changes were observed in NOTCH1 binding or levels of histone modifications H3K27ac, H3K4me1, or H3K4me3 at these loci (FIGS. 12A and 12B), suggesting specific destabilization of Mediator and RNAP2 following LUNAR1 depletion. No changes were observed in the status of any of these factors at the ACTB promoter, again suggesting a locus-specific effect. Because depletion of LUNAR1 led to locus-specific loss of Mediator and RNA PolII binding, it was hypothesized that LUNAR1 RNA might co-occupy the chromatin in those loci. Using chromatin isolation by RNA purification (ChIRP), LUNAR1 RNA was efficiently retrieved (FIG. 12D) and specific enrichment of LUNAR1 was observed at the IGF1R enhancer and LUNAR1 promoter (FIG. 12E), which supports a mechanism by which LUNAR1 exploits chromatin configuration to reach its targets (FIG. 12F) (Engreitz et al., “The Xist lncRNA Exploits Three-dimensional Genome Architecture to Spread Across the X Chromosome,” Science 341:1237973 (2013), which is hereby incorporated by reference in its entirety). Together these data suggest that the intronic IGF1R enhancer activates LUNAR1, which then co-occupies the element and further recruits Mediator in order to sustain full activation of the IGF1R promoter.

These studies revealed LUNAR1 as a regulator of IGF1 signaling and T-ALL cell growth, identifying a putative lncRNA that could be therapeutically targeted in acute leukemia. Additionally, further evidence is provided for the ubiquitous presence and functional importance of lncRNAs in human disease and provides evidence that lncRNAs, in addition to protein-coding genes, are key downstream targets of Notch signaling.

### Discussion of Examples 1-6

The last decades were characterized by an extensive delineation of oncogenic pathways in different cancer types, focusing on protein-coding transcripts and more recently on noncoding miRNA networks. However, very little was known on expression patterns and biological significance of lncRNAs in human tumorigenesis. Also, very little was reported on the regulation of such lncRNAs by well-described oncogenic signaling pathways. Here the expression of lncRNAs in acute leukemia is mapped, using T-ALL, a disease characterized by activation of the NOTCH pathway. Using integration of whole transcriptome analysis and genome-wide chromatin state maps, T-ALL-specific lncRNA genes were systematically identified and noncoding transcripts regulated directly by the binding and the activation of NOTCH1 were characterized. To further suggest biological significance, RNAseq of primary human T ALL was used, characterized by NOTCH1 and FBXW7 mutations (leading to NOTCH activation), and the existence of T-ALL-specific lncRNAs also regulated by NOTCH1 activity was proved, suggesting that NOTCH signaling is able to shape not only the protein coding but also the lncRNA landscape in this disease. These results suggest that—at least a fraction of—the NOTCH1-triggered oncogenic activity could be due to its ability to regulate such noncoding transcripts.

To test this assumption, LUNAR1, a lncRNA that shows T-ALL-specific Notch-dependent expression patterns, is localized in the nucleus, and displays high correlation with IGF1R, a receptor previously suggested to play a role in T-ALL (Medyouf et al., “High-level IGF1R Expression is Required for Leukemia-initiating Cell Activity in T-ALL and is Supported by Notch Signaling,” J. Exp. Med. 208:1809-1822 (2011), which is hereby incorporated by reference in its entirety) was selected. Using assays that can map chromosome conformation and looping, an interaction was shown between the TSS of LUNAR1 and an enhancer element located within the IGF1R locus, characterized by NOTCH1 binding (FIGS. 7 and 8). To prove the connection between LUNAR1 expression and IGF1R, it was demonstrated that the silencing of this lncRNA led to a significant downregulation of IGF1R expression in T-ALL cells and diminished IGF1 pathway activity. Although it is possible that IGF1R is not the sole target of LUNAR1, it is proposed that one of its key functions is to modulate IGF1 signaling in T-ALL by regulation transcription of the receptor gene (FIG. 9).

Using various assays (FIGS. 11 and 12), evidence has been provided that LUNAR1 belongs to a subclass of enhancer-like lncRNAs, which have been reported previously (Lai et al., “Activating RNAs Associate with Mediator to Enhance Chromatin Architecture and Transcription,” Nature 494:497-501 (2013); Ørom et al., “Long Noncoding RNAs with Enhancer-like Function in Human Cells,” Cell 143:46-58 (2010); Wang et al., “A Long Noncoding RNA Maintains Active Chromatin to Coordinate Homeotic Gene Expression,” Nature 472:120-124 (2011b); Yang et al., “Essential Role of lncRNA Binding for WDR5 Maintenance of Active Chromatin and Embryonic Stem Cell Pluripotency,” Elife 3:e02046 (2014), which are hereby incorporated by reference in their entirety). Among enhancer-like lncRNAs, there appear to be two distinct functional mechanisms at play. One mechanism involves lncRNA-dependent recruitment of WDR5-containing methyltransferase complexes, which catalyzed methylation of the tails of histone 3 on lysine 4 (Gomez et al., “The NeST Long ncRNA Controls Microbial Susceptibility and Epigenetic Activation of the Interferon-g Locus,” Cell 152:743-754 (2013); Wang et al., “A Long Noncoding RNA Maintains Active Chromatin to Coordinate Homeotic Gene Expression,” Nature 472:120-124 (2011b); Yang et al., “Essential Role of lncRNA Binding for WDR5 Maintenance of Active Chromatin and Embryonic Stem Cell Pluripotency,” Elife 3:e02046 (2014), which are hereby incorporated by reference in their entirety). A second mechanism involves stabilization of Mediator complexes and RNA PolII at enhancer elements (Lai et al., “Activating RNAs Associate with Mediator to Enhance Chromatin Architecture and Transcription,” Nature 494:497-501 (2013), which is hereby incorporated by reference in its entirety). Based on chromatin experiments following LUNAR1 depletion, this lncRNA is most likely functionally similar to the noncoding RNA activators (nc-RNA-a) that have been described previously (Lai et al., “Activating RNAs Associate with Mediator to Enhance Chromatin Architecture and Transcription,” Nature 494:497-501 (2013); Ørom et al., “Long Noncoding RNAs with Enhancer-like Function in Human Cells,” Cell 143:46-58 (2010), which are hereby incorporated by reference in their entirety). Because there are currently no methods for predicting lncRNA function based on sequence alone, it is believed that these results represent an incremental, yet important contribution to the overall understanding of lncRNA biology.

The mapping of lncRNAs in human T-ALL opens the possibility that such previously uncharacterized transcripts are key modulators of cellular transformation, through their interaction with oncogenic and tumor suppressor programs in leukemia. Additionally, the suggested (and reported also here) tissue and celltype specificity of lncRNA expression would suggest that such transcripts are powerful and specific biomarkers used to categorize cancer subtypes and stratify patients for clinical trials and therapeutic protocols.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A method of treating T cell acute lymphoblastic leukemia in a subject comprising:
selecting a subject having T cell acute lymphoblastic leukemia and
administering, to the selected subject, a therapeutic agent that inhibits a NOTCH-1 regulated long non-coding RNA (lncRNA) at a dosage effective to treat the T cell acute lymphoblastic leukemia in the subject.

2. The method of claim 1, wherein the NOTCH-1 regulated lncRNA is selected from the group of NOTCH regulated lncRNAs listed in Table 1.

3. The method of claim 1, wherein the NOTCH-1 regulated lncRNA is selected from the group consisting of LUNAR1, Linc94, and Lin_CXCR4.

4. The method of claim 1, wherein the therapeutic agent is an antisense RNA, antisense DNA/RNA hybrid oligonucleotide, siRNA, esiRNA, shRNA, or RNA apatamer.

5. The method of claim 4, wherein the therapeutic agent is an antisense DNA/RNA hybrid oligonucleotide comprising the nucleotide sequence of SEQ ID NO: 47, SEQ ID NO: 48, SEQ ID NO: 49, or SEQ ID NO: 50.

6. The method of claim 4, wherein the therapeutic agent is a shRNA comprising the nucleotide sequence of SEQ ID NO: 41, SEQ ID NO: 42, or SEQ ID NO: 45.

7. The method of claim 1, wherein said administering is repeated periodically.

8. The method of claim 1, further comprising:
administering one or more chemotherapeutic agent to the subject in combination with said therapeutic agent.

9. The method of claim 8, wherein the one or more chemotherapeutic agents is selected from the group consisting of cytarabine, vincristine, prednisone, doxorubicin, daunorubicin, PEG asparaginase, methotrexate, cyclophosphamide, L-asparaginase, etoposide, and leucovorin.

10. A method of diagnosing T cell acute lymphoblastic leukemia in a subject comprising:
measuring, in a biological sample obtained from a subject, expression levels of one or more NOTCH-1 regulated lncRNA transcripts;
comparing the measured expression levels to control expression levels of the one or more NOTCH-1 regulated lncRNA transcripts; and
diagnosing the subject as having T cell acute lymphoblastic leukemia based on said comparing.

11. The method of claim 10, wherein the NOTCH-1 regulated lncRNA is selected from the group of NOTCH regulated lncRNAs listed in Table 1.

12. The method of claim 10, wherein the one or more NOTCH-1 regulated lncRNA transcripts is selected from the group consisting of LUNAR1, Linc94, and Lin_CXCR4

13. The method of claim 10, wherein the biological sample comprises leukemia-initiating cells.

14. The method of claim 10, wherein said diagnosing comprises:
identifying a sub-type of T cell acute lymphoblastic leukemia in the subject.

15. The method of claim 14 further comprising:
selecting a suitable therapeutic treatment based on said diagnosing and
administering the selected therapeutic treatment to the subject.

16. The method of claim 15, wherein the subject is diagnosed as having a Notch-mediated form of T cell acute lymphoblastic leukemia based on said comparing, and is administered a Notch receptor agonist based on said diagnosis.

17. The method of claim 10, wherein said measuring is carried out by microarray analysis, polymerase chain reaction, reverse transcriptase polymerase chain reaction, chromatin isolation by RNA purification (ChiRP), serial analysis of gene expression (SAGE), a sequencing reaction, an immunoassay, or mass spectrometry.

18. A kit suitable for diagnosing T-ALL comprising:
one or more reagents suitable for detecting the expression levels of one or more NOTCH-1 regulated lncRNAs.

19. The kit according to claim 18 further comprising:
reagents suitable for isolating T-ALL cells.

20. The kit according to claim 18, wherein the one or more reagents comprise oligonucleotide primers suitable for detecting the expression LUNAR1, Linc94, and Lin_CXCR4, or any combination thereof using a quantitative polymerase chain reaction.

21. The kit according to claim 18, wherein the one or more reagents comprise oligonucleotide probes suitable for detecting the expression of LUNAR1, Linc94, and Lin_CXCR4, or any combination thereof by using a chromatin isolation by RNA purification assay (ChIRP).


SEQUENCE LISTING (74 sequences)

SEQ ID NO: 1 - DNA, 491 bp, Homo sapiens
aggaatgggagagggaggaggacgccaggggcacagggacagcctccggggcggggcgcggcgccagcctagcccgaggatggaggctgaggccgcctgttgagtcacagtttccctgtgatgctcattttaaccagatggcacttgcttagcagtttggaggagaagacctgttcccctgcagccttccatgattagacctcaactcatgagaactcttgccttgctcacaagtgggagtaaatgctcatagctctagaaatctcccaggaaccagacatcacggaaactagaaaggctaagggagtccaatcttcctctaaattggccaacagtagagtggtggaaagagaacaagctgcaaaccagacagacccaagcttgaatctcacctctgccaagctaccagagctcaacaaactacagaccacgggccaaatctgacccaccgctgcttttgtaaataaaactttattggaaaaaaaaaaaaa

SEQ ID NO: 2 - DNA, 1102 bp, Homo sapiens
aggcctttcctgcaacactgagctgtttctttccttttcttttttaaccatgcaacaaaacctttattagcattttgaacaggttcagctattactgaaacttgtaatttctaaacttaagttggggcaaatggctatacggcagagtaatgccatcactgggcactgcgaatgcaagactggagaattaacagccacccctcaggtgcaggaccaggtgcagggttgactctttctggatgttgtagtcagaaagagtgcggccatcttccagctgcttgcctgcaaagatgagcctctgctggtcggggctgggggtgggggggtgccttctttatcctggatcttggccttcacattttccatggtgtcactgggctccacttccagggtgatggtcttgccagtcagggtcttcacgaagatctgcataccacctctcagacacaggaccaggtgcagggtctactctttctggatgttatagtcagaatgagtgcagtcatcttccacctgcttgactgcaaagatgagcctctgctggtccggggtaatgccttccttatcctggatcttggccttcacattttcgatggtgtccctgggctccacttcaagggcaatggtcttgctggtaagggtcttcacgaagatctgcattttgacctgttagcggatatgacgaggctccgaaacaccagtcatgtccagccacagggacaccaccacatactcacccaacaaagccagtcatccctaccactgagctatttctatgcgagttcttcccttggcccttaagctgggataaatccctgtcttcatgcaaagttagagacatgattagatacaagatctacaatatttgtggataaaaaccaaacagttccttaagaaaactacaactattttttttggctgacaccagagtgaaatttcccccatttatcccccatcagcctttggtaggagcacaaaagctacgtggcagggcacattccagcaccatgcccatgacaccaactctcgttcattcattccttgacgtatttacattcaaactccgtcctcgtttgctgctgtgctgctggttctggctcca

SEQ ID NO: 3 - DNA, 574 bp, Homo sapiens
gcgaccttgaagcggcatccgaggagatgtggccacggggcaggcgaccgacaccagcgagtccagagggccagcgtgtgcaccactgtgtgtctccagagacttcaggaagcagccaccacgcccgaggaatgcaggaagatggacacacggctggggaagtacaatgaaaggccaagtaggcagcctgttctcctcagatcagtcccccacgaacactcattcccgaggactcatccaatactaataagagaatgctcttgtttttgaagaattttctgaaagccatcctgacaaattaagtagagtatgctgaagatagtcagactttgtttttaagaattgaatattctggaagaggctcttcagtccaatctttagttctctccacagagcaaacgaagtgaagtgctgaaggcctggagcccgagctgttcccacacggactccaggacagttaaggcagggttgccttaactaaatctctgacaactgtttcttctgtctttctcctaaaaatggaatgcaggccatctattctagggaaataaaggattctagttatgtgaatcca

SEQ ID NO: 4 - DNA, 20 bp, Artificial Sequence
  hGAPDH_F cDNA primer
cttttgcgtcgccagccgag

SEQ ID NO: 5 - DNA, 20 bp, Artificial Sequence
  hGAPDH_R cDNA primer
ccaggcgcccaatacgacca

SEQ ID NO: 6 - DNA, 20 bp, Artificial Sequence
  hIGF1R_F cDNA primer
tactcggacgtctggtcctt

SEQ ID NO: 7 - DNA, 20 bp, Artificial Sequence
  hIGF1R_R cDNA primer
tggggttatactgccagcac

SEQ ID NO: 8 - DNA, 21 bp, Artificial Sequence
  hDTX1_F cDNA primer
cagcttgtgccctacatcatc

SEQ ID NO: 9 - DNA, 20 bp, Artificial Sequence
  hDTX1_R cDNA primer
acgacgggtcgtagaagttg

SEQ ID NO: 10 - DNA, 20 bp, Artificial Sequence
  hHES1_F cDNA primer
gcagatgacggctgcgctga

SEQ ID NO: 11 - DNA, 22 bp, Artificial Sequence
  hHES1_R cDNA primer
aagcgggtcacctcgttcatgc

SEQ ID NO: 12 - DNA, 20 bp, Artificial Sequence
  hU1_F cDNA primer
atacttacctggcaggggag

SEQ ID NO: 13 - DNA, 21 bp, Artificial Sequence
  hU1_R cDNA primer
cagggggaaagcgcgaacgca

SEQ ID NO: 14 - DNA, 20 bp, Artificial Sequence
  LUNAR1_F cDNA primer
ggaggctgaggccgcctgtt

SEQ ID NO: 15 - DNA, 22 bp, Artificial Sequence
  LUNAR1_R cDNA primer
aggctgcaggggaacaggtctt

SEQ ID NO: 16 - DNA, 20 bp, Artificial Sequence
  HOTTIP_F1 cDNA primer
cctaaagccacgcttctttg

SEQ ID NO: 17 - DNA, 20 bp, Artificial Sequence
  HOTTIP_R1 cDNA primer
tgcaggctggagatcctact

SEQ ID NO: 18 - DNA, 20 bp, Artificial Sequence
  linc94 forward cDNA primer
cacggctggggaagtacaat

SEQ ID NO: 19 - DNA, 20 bp, Artificial Sequence
  linc94 reverse cDNA primer
tcgggaatgagtgttcgtgg

SEQ ID NO: 20 - DNA, 20 bp, Artificial Sequence
  hIGF1Renh_F genomic DNA primer
gtggtttagggtgggtgagg

SEQ ID NO: 21 - DNA, 20 bp, Artificial Sequence
  hIGF1Renh_R genomic DNA primer
aaaagcccagtcacctggag

SEQ ID NO: 22 - DNA, 20 bp, Artificial Sequence
  LUNAR1pro_F genomic DNA primer
cgggtgcaccttctgaatct

SEQ ID NO: 23 - DNA, 20 bp, Artificial Sequence
  LUNAR1pro_R genomic DNA primer
tccccacaaggagaagggaa

SEQ ID NO: 24 - DNA, 20 bp, Artificial Sequence
  ACTBpro_F genomic DNA primer
aaaggcaactttcggaacgg

SEQ ID NO: 25 - DNA, 20 bp, Artificial Sequence
  ACTBpro_R genomic DNA primer
ttcctcaatctcgctctcgc

SEQ ID NO: 26 - DNA, 20 bp, Artificial Sequence
  linCXCR4_F genomic DNA primer
tgatggtcttgccagtcagg

SEQ ID NO: 27 - DNA, 20 bp, Artificial Sequence
  linCXCR4_R genomic DNA primer
ttcggagcctcgtcatatcc

SEQ ID NO: 28 - DNA, 25 bp, Artificial Sequence
  LUNAR1_promoterBait probe 3C primer
aggaggaaaattgagggtgactcag

SEQ ID NO: 29 - DNA, 24 bp, Artificial Sequence
  LUNAR1_promoterBait primer
cagggctagtagactagaaagata

SEQ ID NO: 30 - DNA, 25 bp, Artificial Sequence
  IGF1R_enhancerBait probe
agcccatgaaagcaggtgtgagttc

SEQ ID NO: 31 - DNA, 26 bp, Artificial Sequence
  IGF1R_enhancerBait primer
ctcacccaccctaaaccacagaagca

SEQ ID NO: 32 - DNA, 20 bp, Artificial Sequence
  enhancerUs5_R 3C primer
tccatgtgggagagacagtg

SEQ ID NO: 33 - DNA, 20 bp, Artificial Sequence
  enhancerUs4_R 3C primer
tttcagtgaagtcccccatc

SEQ ID NO: 34 - DNA, 21 bp, Artificial Sequence
  enhancerUs3_R 3C primer
cccacctaacaatgatcctga

SEQ ID NO: 35 - DNA, 20 bp, Artificial Sequence
  enhancerDs1_R 3C primer
ccttctcacccatcgatctc

SEQ ID NO: 36 - DNA, 20 bp, Artificial Sequence
  enhancerDs2_R 3C primer
atagccgggcttagcttctc

SEQ ID NO: 37 - DNA, 20 bp, Artificial Sequence
  enhancerDs3_R 3C primer
gcccacctcaggctttactt

SEQ ID NO: 38 - DNA, 20 bp, Artificial Sequence
  enhancerDs4_R 3C primer
gatcagcattgtgagcagca

SEQ ID NO: 39 - DNA, 20 bp, Artificial Sequence
  enhancerDs5_R 3C primer
ggagtccagcacagtgtcaa

SEQ ID NO: 40 - DNA, 26 bp, Artificial Sequence
  control primer
agggctccaatgccaaataagtgtgg

SEQ ID NO: 41 - DNA, 97 bp, Artificial Sequence
  shLUNAR1_1 shRNA
tgctgttgacagtgagcgccagtagagtggtggaaagagatagtgaagccacagatgtatctctttccaccactctactgttgcctactgcctcgga

SEQ ID NO: 42 - DNA, 97 bp, Artificial Sequence
  shLUNAR1_2 shRNA
tgctgttgacagtgagcgcccgcctgttgagtcacagttttagtgaagccacagatgtaaaactgtgactcaacaggcggttgcctactgcctcgga

SEQ ID NO: 43 - DNA, 97 bp, Artificial Sequence
  shMYC shRNA
tgctgttgacagtgagcgccagaatttcaatcctagtatatagtgaagccacagatgtatatactaggattgaaattctgttgcctactgcctcgga

SEQ ID NO: 44 - DNA, 97 bp, Artificial Sequence
  shRenilla shRNA
tgctgttgacagtgagcgcaggaattataatgcttatctatagtgaagccacagatgtatagataagcaataattcctatgtgcctactgcctcgga

SEQ ID NO: 45 - DNA, 97 bp, Artificial Sequence
  shlinc94 shRNA
tgctgttgacagtgagcgccacggactccaggacagttaatagtgaagccacagatgtattaactgtcctggagtccgtgttgcctactgcctcgga

SEQ ID NO: 46 - DNA, 19 bp, Artificial Sequence
  ASO-scr
nnnnncgcaccagcgnnnn

SEQ ID NO: 47 - DNA, 25 bp, Artificial Sequence
  ASO1-LUNAR1 with modified backbone
nnnnnnctcctccaaactgnnnnnn

SEQ ID NO: 48 - DNA, 25 bp, Artificial Sequence
  ASO2-LUNAR1 with modified backbone
nnnnnntagcctttctagtnnnnnn

SEQ ID NO: 49 - DNA, 25 bp, Artificial Sequence
  ASO1-Lin-CXCR4 with modified backbone
nnnnnnggtgagtatgtggnnnnnn

SEQ ID NO: 50 - DNA, 26 bp, Artificial Sequence
  ASO2-Lin-CXCR4 with modified backbone
nnnnnngttgggtgagtatgnnnnnn

SEQ ID NO: 51 - DNA, 20 bp, Artificial Sequence
  LUNAR1_01 ChIRP probe
aaactgtgactcaacaggcg

SEQ ID NO: 52 - DNA, 20 bp, Artificial Sequence
  LUNAR1_02 ChIRP probe
gccatctggttaaaatgagc

SEQ ID NO: 53 - DNA, 20 bp, Artificial Sequence
  LUNAR1_03 ChIRP probe
cctccaaactgctaagcaag

SEQ ID NO: 54 - DNA, 20 bp, Artificial Sequence
  LUNAR1_04 ChIRP probe
gaaggctgcaggggaacagg

SEQ ID NO: 55 - DNA, 20 bp, Artificial Sequence
  LUNAR1_05 ChIRP probe
catgagttgaggtctaatca

SEQ ID NO: 56 - DNA, 20 bp, Artificial Sequence
  LUNAR1_06 ChIRP probe
tgtgagcaaggcaagagttc

SEQ ID NO: 57 - DNA, 20 bp, Artificial Sequence
  LUNAR1_07 ChIRP probe
ctatgagcatttactcccac

SEQ ID NO: 58 - DNA, 20 bp, Artificial Sequence
  LUNAR1_08 ChIRP probe
gttcctgggagatttctaga

SEQ ID NO: 59 - DNA, 20 bp, Artificial Sequence
  LUNAR1_09 ChIRP probe
tttctagtttccgtgatgtc

SEQ ID NO: 60 - DNA, 20 bp, Artificial Sequence
  LUNAR1_10 ChIRP probe
tagaggaagattggactccc

SEQ ID NO: 61 - DNA, 20 bp, Artificial Sequence
  LacZ_01 ChIRP probe
ccagtgaatccgtaatcatg

SEQ ID NO: 62 - DNA, 20 bp, Artificial Sequence
  LacZ_02 ChIRP probe
gtagccagctttcatcaaca

SEQ ID NO: 63 - DNA, 20 bp, Artificial Sequence
  LacZ_03 ChIRP probe
atcttccagataactgccgt

SEQ ID NO: 64 - DNA, 20 bp, Artificial Sequence
  LacZ_04 ChIRP probe
ataatttcaccgccgaaagg

SEQ ID NO: 65 - DNA, 20 bp, Artificial Sequence
  LacZ_05 ChIRP probe
ttcatcagcaggatatcctg

SEQ ID NO: 66 - DNA, 20 bp, Artificial Sequence
  LacZ_06 ChIRP probe
tgatcacactcgggtgatta

SEQ ID NO: 67 - DNA, 20 bp, Artificial Sequence
  LacZ_07 ChIRP probe
aaacggggatactgacgaaa

SEQ ID NO: 68 - DNA, 20 bp, Artificial Sequence
  LacZ_08 ChIRP probe
gttatcgctatgacggaaca

SEQ ID NO: 69 - DNA, 20 bp, Artificial Sequence
  LacZ_09 ChIRP probe
tgtgaaagaaagcctgactg

SEQ ID NO: 70 - DNA, 20 bp, Artificial Sequence
  LacZ_10 ChIRP probe
gtaatcgccatttgaccact

SEQ ID NO: 71 - DNA, 25 bp, Artificial Sequence
  ASO1-LUNAR1 nucleotide sequence
ggucuuctcctccaaactgcuaagc

SEQ ID NO: 72 - DNA, 25 bp, Artificial Sequence
  ASO2-LUNAR1 nucleotide sequence
cucccutagcctttctagtuuccgu

SEQ ID NO: 73 - DNA, 25 bp, Artificial Sequence
  ASO1-Lin-CXCR4 nucleotide sequence
uuguugggtgagtatgtgguggugu

SEQ ID NO: 74 - DNA, 26 bp, Artificial Sequence
  ASO2-Lin-CXCR4 nucleotide sequence
ggcuuugttgggtgagtatguggugg